(19) United States
(12) Patent Application Publication
(10) Pub. No.: US 2009/0291087 A1
(43) Pub. Date: Nov. 26, 2009

(54) MODULATING ANGIOGENESIS

(75) Inventors: Edward W. SCOTT, Gainesville, FL (US); Christopher R. COGLE, Gainesville, FL (US)

Correspondence Address: AKERMAN SENTERFITT, P.O. BOX 3188, WEST PALM BEACH, FL, 33402-3188, US

(73) Assignee: University of Florida Research Foundation, Inc., Gainesville, FL (US)

(21) Appl. No.: 12/358,891

(22) Filed: Jan. 23, 2009

Related U.S. Application Data
(63) Continuation-in-part of application No. 10/392,439, filed on Mar. 18, 2003, now Pat. No. 7,491,389.
(60) Provisional application No. 60/367,078, filed on Mar. 21, 2002.
(60) Provisional application No. 60/429,744, filed on Nov. 27, 2002.
(60) Provisional application No. 60/448,691, filed on Feb. 19, 2003.
(60) Provisional application No. 61/133,899, filed on Jul. 3, 2008.

(51) Int. Cl. A61K 39/395; A61P 35/02; A61P 35/00

(52) U.S. Cl. 424/145.1; 424/158.1

(57) ABSTRACT

Hemangioblasts in adult bone marrow participate in new blood vessel formation. By modulating the differentiation of hemangioblasts into blood vessel cells, angiogenesis in a particular tissue can be increased or decreased. The present invention features compositions and methods for reducing tumor vasculogenesis, treating leukemia, and/or treating or preventing leukemia relapse. In particular, the invention provides an SDF-1 binding agent (e.g., antibody, antisense, ribozyme) for the treatment or prevention of a neoplasia, such as leukemia. Intravitreal injection of antibodies that block SDF-1 activity inhibited induced retinal neovascularization mediated by hemangioblasts. Anti-SDF-1 ribozymes and SDF-1 anti-sense RNA expression constructs significantly reduced migration of cells that create new vessels in the eye.

### CROSS REFERENCE TO RELATED APPLICATION

This application is a continuation-in-part application of U.S. patent application Ser. No. 10/392,439, filed Mar. 18, 2003, and also claims the priority of U.S. provisional patent application Ser. No. 60/367,078, filed Mar. 21, 2002; U.S. provisional patent application Ser. No. 60/429,744 filed Nov. 27, 2002; U.S. provisional patent application Ser. No. 60/448,691 filed Feb. 19, 2003; and U.S. provisional patent application Ser. No. 61/133,899 filed Jul. 3, 2008.

### STATEMENT AS TO FEDERALLY SPONSORED RESEARCH

The invention was made with United States government support under grant numbers HL70738, EY012601, EY007739, CA72769, CA089655, DK52558, DK067359, and R01 HL075258 awarded by the National Institutes of Health, and grant number 05NIR-02-5198 awarded by the Florida Department of Health James & Esther King Biomedical Research Program. The United States government has certain rights in the invention.

### FIELD OF THE INVENTION

The invention relates to the fields of medicine, angiogenesis, and stem cell biology. More particularly, the invention relates to compositions and methods for modulating angiogenesis.

### BACKGROUND

Adult bone marrow (BM)-derived hematopoietic stem cells (HSC) are defined by their ability to self renew while functionally repopulating the cells of the blood and lymph for the life of an individual. See, Müller-Sieburg, C. (ed.) Hematopoietic stem cells: animal models and human transplantation (Springer-Verlag, New York, 1992). These abilities make HSC clinically useful in therapeutic BM transplantation for a variety of BM failure states including the hematological malignancies leukemia and lymphoma. HSC can be highly enriched and quantified by known methods. See, e.g., Harrison et al. Exp Hematol 21, 206-19 (1993). Like other tissue-derived stem cells, HSC are thought to retain a high capacity for “plasticity” that would allow for the potential contribution of regenerative progenitors to non-hematopoietic tissues following injury or stress. Goodell et al., Ann NY Acad. Sci. 938, 208-18; discussion 218-20 (2001); Krause et al., Cell 105, 369-77 (2001).

Indeed, following full and durable reconstitution of a lethally irradiated mouse with a single BM-derived HSC, donor cells were identified in multiple tissues such as the brain, heart, skeletal muscle, liver, and endothelial cells. Krause et al., supra. Although the experimental design of that study yielded low levels (<5%) of donor contribution to non-hematopoietic tissues, the results suggest the possibility of functional regeneration of multiple tissues by HSC-derived progenitors. In other transplant models hematopoietic progenitors have been shown to repopulate hepatocytes in the parenchymal liver to restore liver function following chemically induced injury (Petersen et al., Science 284, 1168-70. (1999); Lagasse et al., Nat Med 6, 1229-34 (2000)), and to regenerate myocardium to improve cardiac function following infarction (Orlic et al., Nature 410, 701-5 (2001)).

Diabetic retinopathy and retinopathy of prematurity are among the leading causes of vision impairment throughout the world. Retinal neovascularization is thought to occur in response to an hypoxic insult which leads to changes in the existing vasculature and compensatory, albeit pathologic, new capillary growth. Grant et al., Diabetes 35, 416-20 (1986); Limb et al., Br J Opthalmol 80, 168-73 (1996). Postnatal neovascularization has been attributed to angiogenesis, a process characterized by sprouting of new capillaries from pre-existing blood vessels. Folkman and Shing, J Biol Chem 267, 10931-4 (1992). Several studies have shown that endothelial progenitor cells (EPC) capable of contributing to in vitro capillary formation can be derived from BM cells. Asahara et al., Science 275, 964-7 (1997); Gehling et al., Blood 95, 3106-12 (2000); Bhattacharya et al., Blood 95, 581-5 (2000); Lin et al., J Clin Invest 105, 71-7 (2000). Pro-angiogenic growth factors such as vascular endothelial growth factor (VEGF, See, e.g., Asahara et al., Embo J 18, 3964-72 (1999); Kalka et al., Circ. Res. 86, 1198-202 (2000)), and granulocyte/macrophage colony stimulating factor (GM-CSF) (see, e.g., Takahashi et al., Nat Med 5, 434-8 (1999)) increase circulating levels of EPC in the adult and promote new blood vessel formation. Recently, it was demonstrated that hydroxymethylglutaryl (HMG)-CoA reductase inhibitors potently augment EPC differentiation by a mechanism involving the angiogenic protein kinase Akt. Dimmeler, et al., J Clin Invest 108, 391-7 (2001). Studies also support the contribution by EPC to blood vessel formation in the adult (Asahara et al., Embo J 18, 3964-72 (1999); Kalka et al., supra; Crosby et al., Circ Res 87, 728-30 (2000); Murohara et al., J Clin Invest 105, 1527-36 (2000)), and in cardiac reperfusion post ischemia (Kocher et al., Nat Med 7, 430-6 (2001); Kawamoto et al., Circulation 103, 634-7 (2001)). However, as these studies were based on short-term transplant and acute injury models, it is not clear whether the cell giving rise to EPCs is the long-term repopulating HSC or other progenitors such as the mesenchymal stem cell.

Within the developing embryo, pluripotent progenitors are generated that are capable of contributing to the formation of blood and blood vessels, a process called hemangiogenesis. Choi, K., Biochem Cell Biol 76, 947-56 (1998); Takakura, et al., Cell 102, 199-209 (2000). These pluripotent stem cells are termed hemangioblasts. Hemangioblasts can also be produced from embryonic stem cells during in vitro differentiation in response to vascular endothelial growth factor. Choi, supra. Heretofore, however, definitive evidence for the existence of the hemangioblast within the adult BM, and in particular for a functional role of such BM-derived cells in new blood vessel formation was lacking.

Blood vessel development is essential to cancer growth and metastasis. Cancers require new blood vessel formation for growth, survival and metastasis. The origin of cancer blood vessels may be from angiogenesis, vessel intussusceptions, vascular mimicry, and/or malignancy-derived. The identification of the source of blood vessel formation in cancer is likely to provide for a therapeutic target for the treatment of cancers, including leukemia. Conventional methods of acute leukemia treatment rely on chemotherapy. However, most patients treated with chemotherapy will suffer from a disease relapse. Methods for treating leukemia and other neoplasias and for preventing relapse are urgently required.

### SUMMARY

The invention relates to the discovery that hemangioblasts can be isolated from adult BM. Isolated hemangioblasts can clonally differentiate into all hematopoietic cell lineages as well as blood and blood vessel cells that revascularize adult retina. Because of their ability to promote neovascularization, adult hemangioblasts contribute to ischemia-induced retinal vascular diseases such as diabetic retinopathy and retinopathy of prematurity. Such cells thus represent a new therapeutic target in the treatment of the diseases associated with angiogenesis. For example, compositions and methods of the invention may be useful for treating and preventing cancerous tumor growth by restricting blood supply. Further due to their ability to promote new vessel growth, the therapeutic potential of hemangioblasts can be applied to any disease where vascular endothelium is defective or has been damaged, e.g., ischemia, such as cardiac ischemia.

The invention also relates to the discovery that hemangioblast-mediated neovascularization can be inhibited by blocking SDF-1 (e.g., SDF-1alpha) activity, e.g., using anti-SDF-1 antibodies, anti-SDF-1 ribozymes, SDF-1 anti-sense RNA. As an example, intravitreal injection of neutralizing anti-SDF-1 antibodies completely blocked hemangioblast-derived neovascularization of ischemic retinas. As another example, bone marrow-derived cancer vasculogenesis in melanoma and lymphoma was observed, and administration of anti-SDF-1 antibodies to rodents having lung cancer inhibited tumor angiogenesis. CD133+CXCR4+cells are derived from hemangioblasts and are circulating progenitors (also referred to as functional endothelial progenitor cells). Anti-SDF-1 treatment reduces and blocks mobilization of the CD133+/CXCR4+hemangioblast-derived endothelial progenitor cells as well as any other CXCR+4-expressing cell type including but not limited to: endothelial cells, lymphocytes, myeloid cells, and hematopoietic stem and progenitor cells. Modulating SDF-1 activity thus might be used to treat or prevent diabetic retinopathy and cancer, as well as other diseases related to aberrant vessel formation.

Accordingly, described herein is a method of reducing blood vessel formation in a neoplasia. The method includes administering to a subject having a neoplasia a composition including an agent that binds SDF-1 and reduces SDF-1 biological activity in an amount effective to inhibit blood vessel formation in the neoplasia. The neoplasia can be one of, for example, lung cancer, pancreatic cancer, melanoma, lymphoma, and leukemia. In one embodiment, the agent that binds SDF-1 and reduces SDF-1 biological activity is an antibody that specifically binds SDF-1. In other embodiments, the agent may be an anti-SDF-1 ribozyme or an SDF-1 anti-sense RNA. In the method, administration of the composition to the subject decreases or halts growth of the neoplasia.

Also described herein is a method of reducing marrow cell mobilization in a subject having received chemotherapy. The method includes administering to the subject being treated for a cancerous tumor at a particular site a composition including an agent that binds SDF-1 and reduces SDF-1 biological activity in an amount effective to decrease or halt mobilization of marrow cells and cells that differentiate from marrow cells to the site after the subject has received chemotherapy. Cells that differentiate from marrow cells can be, for example, CD133+CXCR4+ cells and/or cells having surface expression of CD31 and vWF. The method can further include administering a vascular disrupting agent or an agent that reduces VEGF or Tie-2 biological activity to the subject. The composition can be locally administered to a tumor. Administration can be intratumoral or intravascular. As examples, the composition can be administered between about 7 and 60 days, or between about 1 and 28 days following chemotherapy. The composition can be administered following chemotherapy.

Further described herein is a method of inhibiting cancerous tumor growth in a subject having a cancerous tumor. The method includes administering to the subject an agent that binds to SDF-1 and reduces SDF-1 biological activity in an amount effective to decrease or block growth of the cancerous tumor in the subject. In one embodiment, the agent that binds SDF-1 and reduces SDF-1 biological activity is an antibody that specifically binds SDF-1. In other embodiments, the agent may be an anti-SDF-1 ribozyme or an SDF-1 anti-sense RNA. The cancerous tumor growth can be one of, for example, lung cancer, pancreatic cancer, melanoma, lymphoma, or leukemia. The method can further include administering an agent that inhibits VEGF or Tie-2 biological activity (e.g., bevacizumab). In one embodiment, about 0.05-200 mg/kg of the SDF-1 specific antibody is administered to the subject.

Still further described herein is a kit for the treatment of neoplasia or the prevention of a neoplasia relapse. The kit includes a composition including a pharmaceutically acceptable carrier and SDF-1 antibody that specifically binds SDF-1 and blocks SDF-1 biological activity in an amount effective to inhibit neoplasia angiogenesis, and instructions for use. The kit can further include an agent that inhibits VEGF or Tie-2 biological activity.

Unless otherwise defined, all technical terms used herein have the same meaning as commonly understood by one of ordinary skill in the art to which this invention belongs.

Use of the term “hemangioblast” refers to a pluripotent stem/progenitor cell capable of long-term self-renewal and clonally contributing to the formation of blood vessels.

By the term “angiogenesis” is meant the process of vascularization of a tissue involving the development of new blood vessels.

Use of the term “neovascularization” means the formation of new blood vessels.

Use of the term “differentiation” means the changes from simple to more complex forms undergone by developing cells so that they become more specialized for a particular function.

By “agent” is meant a peptide, polypeptide, polynucleotide, ribonucleotide, antibody or small compound.

As used herein, “adult bone marrow” means bone marrow from a postnatal organism.

By “bone marrow derived cell” is meant any cell type that naturally occurs in bone marrow. Such cells include stromal cells, hematopoietic stem and progenitor cells, osteoblasts, fibroblasts, endothelial cells, and macrophages.

By “blood vessel formation” is meant the dynamic process that includes one or more steps of blood vessel development and/or maturation, such as angiogenesis, vasculogenesis, formation of an immature blood vessel network, blood vessel remodeling, blood vessel stabilization, blood vessel maturation, blood vessel differentiation, or establishment of a functional blood vessel network. Methods for measuring blood vessel formation and maturation are standard in the art and are described, for example, in Jain et al., (Nat. Rev. Cancer 2: 266-276, 2002).

By “chemotherapeutic agent” is meant an agent that is used to kill cancer cells or to slow their growth. Accordingly, both cytotoxic and cytostatic agents are considered to be chemotherapeutic agents.

The term “hematopoietic stem cell” refers to a cell that generates blood cells. Hematopoietic stem cell (HSC) may be isolated from bone marrow, blood, or umbilical cord blood. An HSC is the precursor cell that generates blood cells or following transplantation reinitiates multiple hematopoietic lineages and can reinitiate hematopoiesis for the life of a recipient. (See Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827; Hill, B., et al. 1996.) When transplanted into myeloablated animals or humans, hematopoietic stem cells can repopulate the erythroid, neutrophil-macrophage, megakaryocyte and lymphoid hematopoietic cell pool. In vitro, hematopoietic stem cells can be induced to undergo at least some self-renewing cell divisions and can be induced to differentiate to the same lineages observed in vivo.

By “chemotherapy” is meant the treatment of a neoplasia with agents designed to reduce the survival or proliferation of a neoplastic cell.

By “marrow mobilization” is meant the activation and migration of endothelial progenitor cells from the bone marrow to a site outside of the bone marrow.

By “SDF-1” is meant a stromal cell derived factor polypeptide having at least about 85% amino acid sequence identity to GenBank Accession No. NP_{—}954637. The term “SDF-1” encompasses SDF-1 alpha and SDF-1 beta.

By “SDF-1 biological activity” is meant chemokine activity, the promotion of blood vessel formation, or binding to the CXCR4 receptor.

By “treatment regimen” is meant the method or combination of methods used to decrease or ameliorate the progression, proliferation, metastasis, or severity of a neoplasia. A neoplasia treatment regimen typically includes chemotherapy, hormone therapy, immunotherapy, or radiotherapy.

A “tumor,” as used herein, refers to all neoplastic cell growth and proliferation, whether malignant or benign, and all precancerous and cancerous cells and tissues.

By “vascular disrupting agent” is meant an agent that disrupts an established blood vessel network within a tumour. In contrast, an “angiogenesis inhibitor” reduces the growth of new blood vessels.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

A “therapeutically effective amount” is an amount sufficient to effect a beneficial or desired clinical result. A therapeutically effective amount can be administered in one or more doses. In terms of treatment, an effective amount is an amount that is sufficient to palliate, ameliorate, stabilize, reverse or slow the progression of a cancerous disease (e.g. tumors, dysplaysias, leukemias) or otherwise reduce the pathological consequences of the cancer. A therapeutically effective amount can be provided in one or a series of administrations. In terms of an adjuvant, an effective amount is one sufficient to enhance the immune response to the immunogen. The effective amount is generally determined by the physician on a case-by-case basis and is within the skill of one in the art.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Although methods and materials similar or equivalent to those described herein can be used in the practice or testing of the present invention, suitable methods and materials are described below. All publications, patent applications, patents and other references mentioned herein are incorporated by reference in their entirety. In the case of conflict, the present specification, including definitions will control. The particular embodiments discussed below are illustrative only and not intended to be limiting.

### BRIEF DESCRIPTION OF THE DRAWINGS

FIG. 1 is a series of graphs showing that factors affecting leukocyte trafficking influence marrow contribution to cancer blood vessels. FIG. 1A is a graph showing that tumors grew faster and were larger in the cytokine treated group as compared to controls. In the anti-SDF-1 treated group, tumors grew slower and were smaller than controls. FIG. 1B is a graph showing that lung cancer cells grown in vitro in the presence of escalating doses of anti-SDF-1 antibodies did not show a significant difference in growth. FIG. 1C is a graph showing that decreased microvessel density is observed in anti-SDF-1 treated animals. FIG. 1D is a graph showing that marrow-derived lung cancer endothelial cells are increased in cytokine treated animals and decreased in anti-SDF-1 treated animals.

FIG. 2 is a series of micrographs and a pair of graphs showing hematopoietic stem cells contributed to tumor vasculogenesis. BL/6 mice showed the presence of marrow-derived endothelial cells lining blood vessels in lung cancer (FIG. 2A), melanoma (FIG. 2B), and lymphoma (FIG. 2C). Lung cancers (FIG. 2A) demonstrate marrow-derived cells expressing CD31 and lining lumens. Melanomas (B) and lymphomas (C) demonstrate marrow-derived cells expressing CD31 and lining lumens. Anti-SDF-1 therapy in mice leads to decreased lung cancer growth (D). The anti-SDF-1 mechanism is, in part, related to decreases in microvessel density and marrow-derived blood vessels (E).

FIG. 3 is a series of micrographs and a pair of graphs showing that anti-SDF-1α treatment inhibited intimal hyperplasia and tumor neovascularization. FIGS. 3A and 3B are micrographs showing intimal hyperplasia in DsRed⁺ radiation chimeras that received vein grafts which were harvested after two weeks and examined for marrow-derived contributions to intimal hyperplasia. Significant marrow-derived contributions were detected in all grafts examined (n=10). FIG. 3C and FIG. 3D are micrographs showing the effects on intimal hyperplasia of anti-SDF-1α antibody timed release from pellets that were surgically approximated to vein grafts for the two week experimental period. Upon harvest and sectioning there was little if any evidence for marrow-derived intimal hyperplasia in any graft (n=10). FIG. 3E is a micrograph showing tumor endothelium derived from a single transplanted Gfp⁺ HSC. Green is native Gfp fluorescence as confirmed by spectral confocal analysis. Red is IHC staining for CD31 expression. FIG. 3F is a micrograph showing a confocal image of a tumor vessel from a DsRed⁺ radiation chimera that was implanted subcutaneously with a lung cancer cell line. After two weeks of tumor growth, samples were harvested, fixed, sectioned and stained for the endothelial cell marker CD31. Shown is a confocal image of a typical vessel showing DsRed⁺, CD31⁺ cells of marrow origin incorporated in the vascular lumen. FIG. 3G shows the same tumor sectioned and the vessels demarcated in brown (DAB) by staining with an antibody cocktail for CD31, vWF and MECA-32. Note the extensive vascular network in this typical tumor. FIG. 3H is a micrograph showing the effects of administering an anti-SDF-1α antibody every other day at the site of tumor injection. After two weeks the resulting tumors averaged 20% the size of non-treated control tumors. The small tumors were sectioned and stained as in FIG. 3E-3G. FIGS. 3I and 3J are graphs showing the effect of treatment on the percentage of marrow-derived endothelial cells in lung cancer tumors and tumor microvessel density, respectively. For FIG. 3I, vessels in sections were identified by CD31 staining and the percentage of vessels containing at least one donor-marrow derived endothelial cell was determined. For FIG. 3J, the number of CD31⁺ vessels per square millimeter was quantified for each treatment regime.

FIG. 4 is a graph of illustrating leukemia size change after anti-vascular treatments.

FIG. 5 is a schematic illustration, a series of micrographs, and a graph showing differential BM contribution results from the activation of redundant mechanisms of post-natal neovasculogenesis. In 5a, a retinal injury model utilizes vascular endothelial growth factor (VEGF) overexpression by a recombinant adeno-associated virus type 2 that overexpresses the murine 188 isoform of VEGF-A (rAAV2 VEGF-A 188) and laser-induced ischemic injury to promote robust BM derived neovascularization. DsRed⁺ BM-derived blood vessels are shown (n=6; scale bar: 100 μm). All animals were perfused with 30 mg of FITC-Dextran to show functional vasculature and co-stained with α-SMA to confirm endothelial phenotype (n=6; scale bars: 50 μm). In 5b, LLC-based tumors showed GFP⁺ BM contribution throughout the tumor mass (scale bar: 100 μm) mainly from CD11b⁺ cells (n=5; scale bar: 50 μm). Inset is a representative image showing similar results in mice transplanted with DsRed⁺ BM (n=5; scale bar: 20 μm). In 5c, B16-induced tumors had the lowest levels of GFP⁺ BM contribution in comparison to all other models with little contribution within the tumor mass (scale bar: 100 μm) and no contribution within tumor-associated vasculature assessed via claudin-5 staining (n=5; scale bar: 20 μm). MECA-32 staining demonstrated the presence of blood vessels within B16 tumors that occurred at a density similar to LLC tumors (n=5; scale bar: 100 μm). LLC and B16 tumors are outlined with dashed lines.

FIG. 6 is a series of graphs and micrographs showing that endogenously produced SDF-1α is a trigger for BM contribution to sites of post-natal vasculogenesis. In 6a, SDF-1α expression was also observed in LLC tumors with non-detectable levels seen in B16 tumors. Tumors are outlined by dashed lines (n=5; scale bars: 100 μm). In 6b, ELISA analysis of SDF-1α in the serum of mice inoculated with LLC tumors showed a significant increase in serum levels 7-days following tumor inoculation with levels returning to background by day 11 (n=5; * p<0.05). In 6c-f, following LLC inoculation, mice were treated with intratumoral anti-SDF-1α antibodies to block BM contribution (scale bars: 100 μm). Tumors treated with anti-SDF-1α contained significantly lower numbers of BM-derived cells (6c,d), fewer cells integrating within blood vessel walls (6e) and decreased MVD (6f) compared to control tumors (n=5; * p<0.05). In 6g, animals treated with anti-SDF-1α antibodies (solid diamonds) also generated significantly smaller tumors in comparison to controls (solid circles; n=8; * p<0.05).

FIG. 7 is an illustration of a proposed mechanism of bone marrow contribution. Within BM and other tissues including blood vessels reside cells that are capable of participating in new blood vessel formation. At sites of neovascularization, SDF-1α acts as a regulatory molecule necessary for BM recruitment and participation. The extent of contribution is dependent on the model system. Active sites that do not express SDF-1α are much less prone to BM involvement and undergo neovascularization via a non-BM-derived mechanism.

FIG. 8 is a photograph of an electrophoretic gel and a graph showing anti-SDF-1 ribozyme activity: cleavage reaction vs. SDF-1 mRNA in vitro. FIG. 8A shows the raw data and FIG. 8B shows quantification of the raw data.

FIG. 9 is a graph showing results from a chemotaxis assay.

FIG. 10 is a graph showing results from a chemotaxis assay.

### DETAILED DESCRIPTION

The invention provides hemangioblasts isolated from BM as well as methods and compositions for modulating angiogenesis in a target tissue in a subject. The invention also features prophylactic, therapeutic, and diagnostic compositions and methods that are useful for the treatment of neoplasias. The invention is based, at least in part, on the discovery that hematopoietic stem cells derived from bone marrow contribute to blood vessels within tumors and that the intratumoral injection of anti-SDF-1 antibodies reduced tumor growth rate, reduced tumor size, inhibited neovasculogenesis, and reduced bone marrow-derived contribution to tumor neovessels.

As is reported in more detail below, adult mice were durably engrafted with bone marrow cells from transgenic mice expressing green fluorescent protein. Cancers of the lung, pancreas, skin, and lymphatics were injected into mice transplanted with GFP-expressing bone marrow. The injected cells were allowed to form tumors that were subsequently examined for the presence of GFP positive cells. Bone marrow-derived cells, expressing endothelial surface proteins, lacking hematopoietic surface proteins and abutting vascular lumens, were observed in 0.1% to 25% of cancer blood vessels. These endothelial-like cells were classified as tumor endothelial scar cells. Lung cancers in recipients of single cell and serially transplanted HSCs exhibited clonal, donor-derived endothelial scar cells in 5% of tumor vasculature. To investigate the mechanism of HSC-derived contribution to cancer blood vessels, factors involved in leukocyte trafficking were expected to be important to HSC hemangioblast activity in cancer. G-CSF and SCF, which are involved in leukocyte trafficking, were administered to mice after cancer inoculation. Mice that received G-CSF and SCF demonstrated increased tumor growth and increased marrow-derived contribution to tumor neovessels. Blocking intratumoral SDF-1 inhibited tumor growth rate, size, neovasculogenesis, and marrow-derived contribution to tumor neovessels. These findings suggest that the use of agents that disrupt SDF-1 expression or biological activity are likely to be effective for the treatment of a variety of neoplasias. If desired, anti-SDF-1 agents may be used in combination with other therapies that target neoplastic cells or disrupt marrow recruitment to tumor endothelium. Such combination therapies are likely to be more effective than conventional chemotherapy.

Compositions include a hemangioblast isolated from adult BM. In one method, angiogenesis in a target tissue is modulated by a non-naturally occurring step of modulating the level of differentiation of hemangioblasts to blood vessel cells in the subject. For example, to encourage angiogenesis in an ischemic tissue (e.g., myocardium), the differentiation of hemangioblasts to blood vessel cells can be increased by increasing the number of hemangioblasts in the subject. As another example, to reduce angiogenesis in a target tissue (e.g., a retina after hypoxic insult), the differentiation of hemangioblasts to blood vessel cells can be decreased or blocked by decreasing the number of hemangioblasts in the subject. For instance, as described below, intravitreal injection of anti-SDF-1 antibodies inhibited retinal angiogenesis. As another example, administration of anti-SDF-1 antibodies to rodents having lung cancer inhibited tumor angiogenesis. Accordingly, the methods and compositions of the invention might be used to treat a number of disorders associated with aberrant blood vessel formation.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Conventional methods of gene transfer and gene therapy can also be adapted for use in the present invention. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996. Methods of stem cell transplantation are described herein. Such techniques are generally known in the art and are described in detail in Hematopoietic stem cells: animal models and human transplantation, ed. Muller-Sieburg, C., Springer Verlag, NY 1992.

### Hemangioblasts

The invention provides hemangioblasts isolated from adult BM. Hemangioblasts of the invention are HSC that are a source of more differentiated and developmentally restricted progenitors that lack the ability of long-term self-renewal, for example circulating endothelial progenitor cells found in the peripheral blood. The source of the BM from which hemangioblasts are isolated may be from any suitable animal, i.e., any animal having BM containing hemangioblasts. For use in various methods of the invention, the source of BM may be from a non-adult organism, e.g., an embryo. BM can be isolated from an animal using any suitable method. For example, BM may be isolated by needle aspiration of marrow directly from the bone. Hemangioblasts may be isolated from BM using markers differentially expressed on hemangioblasts compared to other BM cells. For example, in human BM, hemangioblasts are positive for marker CD34, and negative for markers CD38 and Lin. Additionally, human hemangioblasts may be isolated using the AC133 marker, or other markers of hematopoietic stem cells. Many different techniques for isolating cells based on marker expression are known, e.g., antibody-based methods, such as immunopanning, magnetic bead separation, and fluorescence activated cell sorting (FACS). As a specific example, BM harvested from a rodent donor is made into a single cell suspension and plated onto tissue culture dishes in IMDM+20% fetal bovine serum (FBS) for 4 hours. Non-adherent cells are collected and several rounds (e.g., three rounds) of lineage antibody depletion (B220, CD3, CD4, CD8, CD11b, Gr-1, TER 119) are performed with a suitable cell sorting system (e.g., Miltyni MACS system) until a small aliquot stained with PE-conjugated lineage antibody cocktail shows greater than approximately 95% lineage-negative by FACS. The Lin− cells are then positively selected for Sca-1 for 2-3 rounds until an aliquot showing greater than approximately 95% Sca-1+, Lin− purity has been achieved. The Sca-1+, Lin− cells are then stained for CD45 to confirm hematopoietic origin. Similar hematopoietic stem cell enrichments are possible for humans to isolate CD34+, CD38−, Lin− cells via either magnetic bead or flow cytometric separation techniques.

Hemangioblasts useful in methods of the invention may include a nucleic acid encoding a detectable label (e.g., green fluorescent protein (GFP)). For example, hemangioblasts containing a nucleic acid encoding GFP are easily visualized by a green fluorescence and are particularly useful in settings where it is desirable to detect the cells as well as daughter cells in newly formed vasculature. Specifically, hemangioblasts may contain a nucleic acid harboring a strong promoter and enhancer (e.g., chicken beta-actin promoter and CMV immediate early enhancer) operably linked to a nucleotide sequence encoding GFP. A description of the gfp+ transgenic mouse strain used as the donor strain in experiments described herein is in Example 1.

Besides maintaining hematopoiesis, primitive cells derived from the bone marrow possess the ability to differentiate into endothelial cells of the vasculature. Endothelial progenitor cells (EPCs) and hematopoietic stem cells (HSCs) are believed to originate from a common hemangioblastic precursor during embryonic development. The adult hematopoietic stem cell is capable of providing hemangioblast activity. In addition, evidence suggests that leukemia cells exhibit hemangioblast activity as well, because endothelial cells harboring the BCR-ABL gene fusion have been detected in patients with CML. In B-cell lymphoma patients, lymphoma-specific genetic alterations were similarly found in endothelial cells comprising the microvasculature.

Based on the results reported herein, leukemia stem cells (LSCs) are a source for leukemic endothelial cells, which suggests that blood vessels may be a sanctuary site for later leukemia relapse. This suggests that therapies for leukemia treatments that include adjuvant anti-vascular therapy will be superior to existing chemotherapy. Accordingly, the invention provides a therapeutic combination that includes angiosuppressive medications in combination with chemotherapy.

### SDF-1

SDF-1 acts as a chemokine in normal and malignant hemangioblast function. When SDF-1 is blocked, a loss of marrow-derived neovasculogenesis is observed. Vascular endothelial growth factor (VEGF) is a heparin-binding cytokine that promotes the proliferation and survival of endothelial cells and hematopoietic stem/progenitor cells. An autocrine loop between VEGF and VEGF receptor 2 (VEGFR2) is critical to hematopoietic stem cell survival, as well as leukemia cell proliferation and survival. Leukemia patients have been treated with the anti-VEGF therapy, bevacizumab (Avastin, Genentech, San Francisco, Calif., USA). Bevacizumab is a recombinant humanized IgG monoclonal antibody directed against VEGF that blocks VEGF binding to its cognate receptors. Based on the results reported herein, a combination of anti-vascular and anti-SDF-1 agents are expected to provide for superior cancer treatment. Combinations of the invention provide for neovasculogenesis inhibition, anti-VEGF therapy, vascular disrupting agents, and Tie-2 inhibitors. Agents to be used include bevacizumab (targeting VEGF), ZD6474 (targeting VEGF receptor tyrosine kinase activity), anti-SDF-1 antibodies (targeting leukemia hemangioblast activity via inhibition of EPC migration), vascular disrupting agents like combrestatin and Oxi4503, and Tie-2 inhibitors like AMG-386. Blocking SDF-1 chemoking signaling in addition to the antivascular effects of bevacizumab, ZD6474, combrestatin, Oxi4503 or AMG-386 is expected to provide a potent anti-leukemic therapy.

### SDF-1 Antagonists and Inhibitors

Intratumoral injections of antibodies against SDF-1 inhibited tumor growth rate, reduced tumor size, reduced neovasculogenesis, and reduced bone marrow-derived contribution to tumor neovessels. In view of these discoveries, described herein are therapeutic agents that inhibit the expression or activity of SDF-1, as well as methods for the use of such agents for the treatment of prevention of a neoplasia. In one embodiment, the invention provides for agents that bind to and block the activity of SDF-1. Such agents include but are not limited to anti-SDF-1 antibodies, aptamers, ribozymes, and antisense molecules.

Methods of constructing and using ribozymes and antisense molecules are known in the art (e.g., Isaka Y., Curr Opin Mol Ther vol. 9:132-136, 2007; Sioud M. and Iversen P. O., Curr Drug Targets vol. 6:647-653, 2005; Ribozymes and siRNA Protocols (Methods in Molecular Biology) by Mouldy Sioud, 2^{nd} ed., 2004, Humana Press, New York, N.Y.). An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire SDF-1 coding strand, or to only a portion thereof. In another embodiment, the antisense nucleic acid molecule is antisense to a “noncoding region” of the coding strand of a nucleotide sequence encoding SDF-1 (e.g., the 5′ and 3′ untranslated regions). Anti-sense agents can include, for example, from about 8 to about 80 nucleobases (i.e. from about 8 to about 80 nucleotides), e.g., about 8 to about 50 nucleobases, or about 12 to about 30 nucleobases. Anti-sense compounds include ribozymes, external guide sequence (EGS) oligonucleotides (oligozymes), and other short catalytic RNAs or catalytic oligonucleotides which hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

### Modulating Angiogenesis of a Target Tissue

Also included within the invention is a method of modulating angiogenesis of a target tissue in a subject. The method includes a non-naturally occurring step of modulating the level of differentiation of hemangioblasts to blood vessel cells in the subject. The target tissue may be any tissue in which it is desired to modulate angiogenesis (e.g., ocular, cardiac, limb, or central nervous system tissue).

The level of differentiation of hemangioblasts to blood vessel cells in the subject may be modulated by a number of methods. In one such method, the number of hemangioblasts in the subject is increased or decreased. The number of hemangioblasts in the target tissue of the subject may be increased by any suitable method, including transplantation of hemangioblasts removed from a donor animal into the subject. The donor can be the subject itself or another animal. In one example of such a method, BM cells are first removed from a donor. Hemangioblasts isolated from the population of BM cells are then cultured in vitro under conditions that allow expansion (e.g., proliferation) of the hemangioblasts. Such conditions generally involve growth of the cells in basal medium containing one or more growth factors (e.g., VEGF, SDF-1). Methods of expanding stem cells in vitro are described in T. Asahara Science 275:964-967, 1997. The expanded cells are then administered to the subject. Several approaches may be used for the reintroduction of hemangioblasts into the subject, including catheter-mediated delivery I.V., or direct injection into the heart, brain, or eye. Techniques for the isolation of donor stem cells and transplantation of such isolated cells are known in the art. Autologous as well as allogeneic cell transplantation may be used according to the invention. Alternatively, the number of hemangioblasts in the target tissue of the subject may be increased by administering factors such as VEGF and GM-CSF that increase circulating levels of blood vessel progenitor cells to promote vessel formation. Molecules such as TNF and NO inhibitors may be used in compositions and methods of the invention to decrease hemangioblast self-renewal, and thereby increase differentiation of hemangioblasts into blood vessel cells.

The number of hemangioblasts in the target tissue of the subject may also be decreased by a number of techniques, including administering to the subject an antibody that specifically binds hemangioblasts. Additionally, the number of hemangioblasts in the target tissue of the subject may be decreased by blocking or decreasing recruitment of hemangioblasts from the BM to a non-BM compartment (e.g., target tissue). This may be accomplished by administering to the subject an agent that decreases or prevents recruitment of hemangioblasts from the BM including antibody that specifically binds SDF-1, heparin derivatives (Presta et al., Curr. Pharm. Des. 9:553-566, 2003), inhibitors that target VEGF and its receptors (e.g., anti-VEGF monoclonal antibody, Jain R K, Semin. Oncol. 29(6 suppl. 16):3-9, 2002), and other integrin, selectin, or adhesion molecules that play a role in hemangioblast or leukocyte migration.

In another method of modulating differentiation of hemangioblasts to blood vessel cells, the recruitment or movement of hemangioblasts from the BM to a non-BM compartment (e.g., target tissue) is increased or decreased. A number of substances are known to increase or decrease recruitment of hemangioblasts. Depending on the particular application, any of these might be used in the invention. To increase recruitment of hemangioblasts from the BM to a non-BM compartment (e.g., the target tissue), the administration of any agent capable of promoting recruitment of hemangioblasts may be used. A number of such agents are known. See, e.g., those described in International Application WO 00/50048; SDF-1 alpha, SDF-1 alpha receptor, integrins (e.g., α4, α5), selectin family of adhesion molecules, and colony stimulating factors such as G-CSF. Additionally, the modulation of endogenous factors that increase hemangioblast recruitment may be useful in promoting hemangioblast recruitment from the BM to a non-BM compartment (e.g., target tissue). For example, SDF-1 alpha is a ligand for CXCR4 and has been shown to induce endothelial cell chemotaxis and to stimulate angiogenesis. Thus, to increase recruitment of hemangioblasts from the BM to a non-BM compartment (e.g., target tissue), SDF-1 levels and/or activity can be increased.

Conversely, reducing or blocking SDF-1 activity can be used in a method of decreasing recruitment of hemangioblasts from BM to a non-BM compartment (e.g., target tissue). The level of SDF-1 activity in the subject may be modulated by decreasing the number of SDF-1 molecules available for binding to a SDF-1 binding molecule (e.g., SDF-1 receptor), for example. An antibody that specifically binds to a SDF-1 polypeptide can be administered to the subject to decrease the number of SDF-1 molecules (e.g., polypeptides) available for binding to the SDF-1 receptor, resulting in the prevention or reduction of recruitment of hemangioblasts from BM to a non-BM compartment (e.g., target tissue). In one example of blocking retinal angiogenesis, an antibody that specifically binds SDF-1 is administered to the eye of a subject. The blocking of hemangioblast recruitment from the BM to a non-BM compartment (e.g., target tissue) can also be achieved by administering to the subject other agents that decrease or block migration of hemangioblasts from BM as described above. For example, an antibody against integrins (e.g., α4, α5), selectin family of adhesion molecules, or colony stimulating factors such as G-CSF can be employed.

In methods of increasing the differentiation of hemangioblasts to blood vessel cells, a number of approaches may be employed. For example, alone or in conjunction with hemangioblast transplantation, an agent that is a positive regulator of hemangioblast differentiation (e.g., cytokines, growth factors) may be upregulated or administered to the subject. For example, cytokines that are negative regulators of hemangioblast self-renewal such as TNF (see e.g., Dybedal et al. Blood 98: 1782-91 (2001)) may be administered to the subject to promote differentiation of hemangioblasts. In particular, chemokines, a large family of inflammatory cytokines, have been shown to play a critical role in the regulation of angiogenesis. A number of angiogenesis assays are commonly utilized to screen the angiogenic or anti-angiogenic activity of chemokines. These include in vitro endothelial cell activation assays and ex vivo or in vivo models of neovascularization. The effect of chemokines on endothelium can be assessed by performing in vitro assays on purified endothelial cell populations or by in vivo assays (Bernardini et al., J. Immunol. Methods 273:83-101, 2003). Regulation of angiogenesis by cytokines is reviewed in Naldini et al., Cur. Pharm. Dis. 9:511-519, 2003.

Molecules such as interleukins, interferons, matrix metalloproteinases, and angiopoietin proteins may also be used as agents for modulating (e.g., increasing) angiogenesis in a subject. Nitrous Oxide (NO) is a key regulator of hemangioblast activity. Accordingly, pharmaceuticals such as sildenafil, amino guanidine, L-name, L-nil and AMT that affect NO levels or inhibit the genes that produce NO can also modulate hemangioblast activity by either blocking/promoting recruitment or altering the size and branch structure of the newly formed vessel.

Growth factors such as fibroblast growth factor (FGF), GM-CSF and transforming growth factor β (TGFβ), and VEGF are also useful for promoting differentiation of hemangioblasts and promoting angiogenesis. Such growth factors act by increasing circulating levels of endothelial progenitor cells to promote new blood vessel formation. A review of growth factors and their receptors in proliferation of microvascular endothelial cells may be found in Suhardja and Hoffman Microsc. Res. Tech. 60:70-75, 2003. Erythropoietin (epo), another pro-angiogenic molecule, has been shown to act synergistically with several growth factors (SCF, GM-CSF, IL-3, and IGF-1) to cause maturation and proliferation of erythroid progenitor cells (Fisher J W, Exp. Biol. Med. 228:1-14, 2003). In a preferred embodiment of increasing angiogenesis, VEGF is administered to the subject to increase differentiation of hemangioblasts and angiogenesis. The VEGF family of growth factors are glycoproteins that are endothelial cell-specific mitogens. VEGF has been shown to stimulate proliferation of endothelial cells and to accelerate the rate at which endothelial cells regenerate. The role of VEGF in angiogenesis is reviewed in Goodgell D S, Oncologist 7:569-570, 2002.

The delivery of a molecule that modulates angiogenesis (e.g., VEGF) can be accomplished using a number of recombinant DNA and gene therapy technologies, including viral vectors. Preferred viral vectors exhibit low toxicity to the host and produce therapeutic quantities of a molecule that modulates angiogenesis. Viral vector methods and protocols are reviewed in Kay et al., Nature Medicine 7:33-40, 2001. Viral vectors useful in the invention include those derived from Adeno-Associated Virus (AAV). A preferred AAV vector comprises a pair of AAV inverted terminal repeats which flank at least one cassette containing a promoter which directs expression operably linked to a nucleic acid encoding a molecule that modulates angiogenesis. Methods for use of recombinant AAV vectors are discussed, for example, in Tal, J., J. Biomed. Sci. 7:279-291, 2000 and Monahan and Samulski, Gene Therapy 7:24-30, 2000.

Useful promoters can be inducible or constitutively active and include, but are not limited to: the SV40 early promoter region (Bemoist et al., Nature 290:304, 1981); the promoter contained in the 3′ long terminal repeat of Rous sarcoma virus (Yamamoto et al., Cell 22:787-797, 1988); the herpes thymidine kinase promoter (Wagner et al., Proc. Natl. Acad. Sci. USA 78:1441, 1981); or the regulatory sequences of the metallothionein gene (Brinster et al., Nature 296:39, 1988).

Several nonviral methods for introducing a nucleic acid encoding a molecule that modulates angiogenesis are also useful in the invention. Techniques employing plasmid DNA for the introduction of a nucleic acid encoding a molecule that modulates angiogenesis (e.g., VEGF) are generally known in the art and are described in references such as Ilan, Y. Curr. Opin. Mol. Ther. 1:116-120, 1999; and Wolff, JA. Neuromuscular Disord. 7:314-318, 1997. Methods involving physical techniques for the introduction of a molecule that modulates angiogenesis into a host cell can be adapted for use in the invention. Such methods include particle bombardment and cell electropermeabilization. Synthetic gene transfer molecules that form multicellular aggregates with plasmid DNA are also useful. Such molecules include polymeric DNA-binding cations (Guy et al., Mol. Biotechnol. 3:237-248, 1995), cationic amphiphiles (lipopolyamines and cationic lipids, Felgner et al., Ann. NY Acad. Sci. 772:126-139, 1995), and cationic liposomes (Fominaya et al., J. Gene Med. 2:455-464, 2000).

In a preferred method of administering an agent that is a positive regulator of hemangioblast differentiation, a nucleic acid encoding a VEGF polypeptide contained within an AAV vector is administered to the subject. The AAV vector may be contained within an AAV particle.

To decrease the level of differentiation of hemangioblasts to blood vessel cells, an agent that is a negative regulator of hemangioblast differentiation may be administered to the subject. Any agent that decreases or blocks differentiation of hemangioblasts to blood vessel cells may be used. Such agents include cytokines, transcription factors such as PU.1, hoxB4 and wnt-5A, or VEGF-receptor agonists. The delivery of cytotoxic antibodies that specifically bind and kill hemangioblasts can be used to block differentiation. Alternatively, cytokines that negatively regulate differentiation may be delivered to the host. Specific immunoglobulin therapy can be used to block molecules such as SDF-1 or CXCR-4 (SDF-1 receptor), α4 and α5 integrins, or the selectin family of adhesion molecules thus preventing recruitment of hemangioblasts to sites of retinal ischemic injury, for example. Additionally, hemangioblast recruitment regimens such as administration of G-CSF can be used to alter hemangioblast activity.

Examples of additional molecules that inhibit or mitigate angiogenesis include dopamine agonists and glucocorticoids (Goth et al., Microsc. Res. Tech. 60:98-106, 2003), endostatin (Ramchandran et al., Crit. Rev. Eukaryot. Gene Expression 12:175-191, 2002), sulfonamides and sulfonylated derivatives (Casini et al., Curr. Cancer Drug Targets 2:55-75, 2002), active site inhibitors of urokinase plasminogen activator (Mazar A P, Anticancer Drugs 12:387-400, 2001), chemokines (Bernardini et al., J. Immunol. Methods 273:83-101, 2003), and somatostatin analogues (Garcia de la Torre et al., Clin. Endocrinol. 57:425-441, 2002), and steroids such as triamcinolone. Additionally, agents that inhibit the expression and/or activity of VEGF and VEGF receptors are useful for modulating (e.g., decreasing) angiogenesis in a subject (Sepp-Lorenzino and Thomas, Expert Opin. Investig. Drugs 11: 1447-1465, 2002). A review of agents that inhibit angiogenesis at the endothelial cell level is found in Jekunen and Kairemo, Microsc. Res. Tech. 60:85-97, 2003.

The delivery and activity of agents for modulating angiogenesis can be enhanced using any of a number of techniques that target delivery to the vasculature as well as compositions with which to manipulate angiogenesis. For example, a nucleic acid encoding an agent for modulating, such as VEGF or SDF-1, can be linked to an endothelial-specific gene for targeting of the agent to the vasculature. A number of drugs are known that promote angiogenesis, and may be useful in compositions of the invention. For a review of recent advances in angiogenesis and vascular targeting, see Bikfalvi and Bicknell, Trends Pharmacol. Sci. 23:576-582, 2002. The administration and/or recruitment of mast cells, which have been shown to promote angiogenesis (Hiromatsu and Toda, Microsc. Res. Tech. 60:64-69, 2003), may be useful in increasing angiogenesis in a subject.

### Isolating Hemangioblasts from Bone Marrow

The invention provides a method for isolating a hemangioblast from the BM of an adult animal. This method includes the steps of isolating bone marrow from the animal, the bone marrow including at least one hemangioblast and at least one non-hemangioblast cell; separating the at least one hemangioblast and the at least one non-hemangioblast cell; and collecting the at least one hemangioblast. Hemangioblasts can be separated from non-hemangioblast cells by any suitable method. In one example of such a method, the BM is contacted with an immobilized agent that specifically binds hemangioblasts but not non-hemangioblast cells. Alternatively, the BM can be contacted with an immobilized agent that specifically binds non-hemangioblast cells but not hemangioblasts. In one variation of these methods, the agent that specifically binds hemangioblasts or non-hemangioblast cells is an antibody.

### Uses for Modulating Angiogenesis

Many applications exist for which methods and compositions of modulating angiogenesis would be useful. Compositions and methods of the invention for increasing angiogenesis in a subject may be useful for treating any vasculature-related disorder in which the absence of vasculature causes or is involved in the pathology of the disorder. Examples of such disorders include anemia, ischemia (e.g., limb ischemia, cardiac and brain ischemia), coronary artery disease, and diabetic circulatory deficiencies.

Examples of physiologic states that would also benefit from angiogenesis provided by compositions and methods of the invention include organ and tissue regeneration, wound healing, and bone healing. Angiogenesis is critical to wound repair (Li et al., Microsc. Res. Tech. 60:107-114, 2003). Newly formed blood vessels participate in provisional granulation tissue formation and provide nutrition and oxygen to growing tissues. In addition, inflammatory cells require the interaction with and transmigration through the endothelial basement membrane to enter the site of injury. Among the most potent angiogenic cytokines in wound angiogenesis are VEGF, angiopoietin, FGF, and TGF-β. Administration of such cytokines in conjunction with administration of hemangioblasts of the invention, therefore, would be useful in promoting wound repair.

Increasing angiogenesis using compositions and methods of the invention is useful for treating ischemic conditions. The ability to develop collateral vessels represents an important response to vascular occlusive diseases (e.g., ischemia). Compositions involving hemangioblasts and angiogenic growth factors may be useful for treating subjects with critical limb ischemia as well as myocardial ischemia. Despite continued advances in the prevention and treatment of coronary artery disease (e.g., myocardial ischemia), there remains a population of patients who are not candidates for the conventional revascularization techniques of balloon angioplasty and stenting, or coronary artery bypass grafting. Angiogenesis of ischemic cardiac tissue or skeletal muscle using compositions and methods of the invention may be used to achieve therapeutic angiogenesis in these and other patients. Recent studies have established the feasibility of using angiogenic growth factors such as VEGF and FGF to enhance angiogenesis in patients with limb or myocardial ischemia (Silvestre and Levy, Vale et al., J. Interv. Cardiol. 14:511-528, 2001).

Compositions and methods of decreasing angiogenesis according to the invention can also serve as an effective therapy for such disorders as diabetic retinopathy. Diabetic retinopathy is a major public health problem and it remains the leading cause of blindness in people between 20 and 65 years of age. Like other blinding diseases, diabetic retinopathy is related to an aberrant angiogenic response (reviewed in Garnder et al., Surv. Ophthalmol. 47 (suppl. 2):S253-262, 2002; and Spranger and Pfeiffer, Exp. Clin. Endocrinol. Diabetes 109 (suppl. 2):S438-450, 2001). In one example of a method of treating diabetic retinopathy, antibodies specific to SDF-1 alpha are administered to a patient, resulting in prevention of angiogenesis.

Additionally, compositions and methods of the invention may be useful for treating and preventing cancerous tumor growth by restricting blood supply. Uncontrolled endothelial cell proliferation is observed in tumor neovascularization and in angioproliferative diseases. Cancerous tumors cannot grow beyond a limited mass unless a new blood supply is provided. Control of the neovascularization process, therefore, represents a therapeutic modality for malignant tumors. Solid-tumor cancers that may be treated using compositions and methods of the invention include gliomas, colorectal carcinomas, ovarian and prostate cancer tumors.

### Mammalian Subjects, Target Tissues, Target Cells and Stem Cells

The invention provides compositions and methods involving modulating angiogenesis of a target tissue in a subject by modulating differentiation of hemangioblasts to blood vessel cells and modulating hemangioblast recruitment to a target tissue of a subject (e.g. mammalian). Mammalian subjects include any mammal such as human beings, rats, mice, cats, dogs, goats, sheep, horses, monkeys, apes, rabbits, cattle, etc. The mammalian subject can be in any stage of development including adults, young animals, and neonates. Mammalian subjects also include those in a fetal stage of development. Target tissues can be any within the mammalian subject such as retina, liver, kidney, heart, lungs, components of gastrointestinal tract, pancreas, gall bladder, urinary bladder, the central nervous system including the brain, skin, bones, etc.

### Transplanting Isolated Hemangioblasts and BM into a Subject

The cells, compositions and methods of the invention can be used to generate as well as regenerate vasculature in a subject (e.g., humans) by cell transplantation. To generate or regenerate vasculature in a subject, cells may be transplanted into a subject by any suitable delivery method. In one method, cells are isolated from a donor animal. Hemangioblasts are isolated from the BM cells and then introduced into the subject. Several approaches may be used for the introducing of hemangioblasts into a subject, including catheter-mediated delivery of I.V., or direct injection into a target tissue, e.g., heart, brain or eye.

Hemangioblasts isolated from BM can be administered to a subject (e.g., a human subject suffering from vascular damage) by conventional techniques. For example, hemangioblasts may be administered directly to a target site (e.g., a limb, myocardium, brain) by, for example, injection (of cells in a suitable carrier or diluent such as a buffered salt solution) or surgical delivery to an internal or external target site (e.g., a limb or ventricle of the brain), or by catheter to a site accessible by a blood vessel. For exact placement, the cells may be precisely delivered into brain sites by using stereotactic injection techniques.

The cells described above are preferably administered to a subject (e.g., mammal) in an effective amount, that is, an amount effective capable of producing a desirable result in a treated subject (e.g., modulating angiogenesis in a subject). Such therapeutically effective amounts can be determined empirically. Although the range may vary considerably, a therapeutically effective amount is expected to be in the range of 500-10⁶ cells per kg body weight of the animal.

### SDF-1 Alpha-Specific Antibodies

The invention relates to modulating the level of SDF-1 activity in a subject by administering to the subject an antibody that specifically binds to a SDF-1 polypeptide. Antibodies that selectively bind a SDF-1 polypeptide are useful in the methods of the invention. Binding to the SDF-1 polypeptide reduces SDF-1 biological activity as assayed by analyzing binding to the CXCR4 receptor. Methods of preparing antibodies are well known to those of ordinary skill in the science of immunology. As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv), fusion polypeptides, and unconventional antibodies.

In one embodiment, an antibody that binds an SDF-1 polypeptide is monoclonal. Alternatively, the anti-SDF-1 antibody is a polyclonal antibody. The preparation and use of polyclonal antibodies are known to the skilled artisan. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′)₂” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab′ fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing SDF-1, or immunogenic fragments thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding a SDF-1 polypeptide or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding a SDF-1 polypeptide, or immunogenic fragments thereof, can be expressed in cells in vitro, followed by isolation of the polypeptide and administration of the polypeptide to a suitable host in which antibodies are raised.

Alternatively, antibodies against a SDF-1 polypeptide may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

In other embodiments, the invention provides “unconventional antibodies.” Unconventional antibodies include, but are not limited to, nanobodies, linear antibodies (Zapata et al., Protein Eng. 8(10): 1057-1062, 1995), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity to cancer cells. These multimeric scFvs (e.g., diabodies, tetrabodies) offer an improvement over the parent antibody since small molecules of ˜60-100 kDa in size provide faster blood clearance and rapid tissue uptake See Power et al., (Generation of recombinant multimeric antibody fragments for tumor diagnosis and therapy. Methods Mol Biol, 207, 335-50, 2003); and Wu et al. (Anti-carcinoembryonic antigen (CEA) diabody for rapid tumor targeting and imaging. Tumor Targeting, 4, 47-58, 1999).

Various techniques for making unconventional antibodies have been described. Bispecific antibodies produced using leucine zippers are described by Kostelny et al. (J. Immunol. 148(5):1547-1553, 1992). Diabody technology is described by Hollinger et al. (Proc. Natl. Acad. Sci. USA 90:6444-6448, 1993). Another strategy for making bispecific antibody fragments by the use of single-chain Fv (sFv) diners is described by Gruber et al. (J. Immunol. 152:5368, 1994). Trispecific antibodies are described by Tutt et al. (J. Immunol. 147:60, 1991). Single chain Fv polypeptide antibodies include a covalently linked VH::VL heterodimer which can be expressed from a nucleic acid including V_{H}- and V_{L}-encoding sequences either joined directly or joined by a peptide-encoding linker as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754.

In addition to modulating the level of SDF-1 activity in a subject, antibodies useful in the invention can also be used, for example, in the detection of a SDF-1 alpha protein (or SDF-1 alpha protein receptor) in a biological sample, e.g., a retina section or cell. Antibodies also can be used in a screening assay to measure the effect of a candidate compound on expression or localization of SDF-1 alpha protein or SDF-1 alpha protein receptor. Additionally, such antibodies can be used to interfere with the interaction of a SDF-1 alpha protein and other molecules that bind the SDF-1 alpha protein such as a SDF-1 alpha protein receptor.

### Screening Assays

The invention provides methods for treating a neoplasia or preventing a relapse of neoplasia following remission. While the Examples described herein specifically discuss the use of an antibody that specifically bind to SDF-1, one skilled in the art understands that the methods of the invention are not so limited. Virtually any agent that specifically binds to SDF-1 and blocks its biological activity may be employed in the methods of the invention.

Methods of the invention are useful for the high-throughput low-cost screening of candidate agents that bind to SDF-1. A candidate agent that specifically binds to SDF-1 is then isolated and tested for activity in an in vitro assay or in vivo assay for its ability to block SDF-1 biological activity. One skilled in the art appreciates that the effects of a candidate agent on a cell or tissue is typically compared to a corresponding control cell not contacted with the candidate agent. Thus, the screening methods include comparing the agents that bind to SDF-1 for their effect on tumor growth rate, tumor size, neovasculogenesis, or bone marrow-derived contribution to tumor neovessels in a cell, tissue, or animal contacted by a candidate agent to the parameters in an untreated control cell, tissue, or animal. Compounds that reduce tumor growth rate, tumor size, neovasculogenesis, or bone marrow-derived contribution to tumor neovessels in a cell by at least about 5%, 10%, 25%, 50%, 75% or more are considered useful in the invention.

In one embodiment, the efficacy of a candidate agent is dependent upon its ability to interact with an SDF-1 polypeptide. Such an interaction can be readily assayed using any number of standard binding techniques and functional assays (e.g., those described in Ausubel et al., supra). For example, a candidate compound may be tested in vitro for interaction and binding with an SDF-1 polypeptide of the invention and its ability to reduce tumor vasculogenesis may be assayed by any standard assays (e.g., those described herein).

Potential SDF-1 binding agents or SDF-1 antagonists include organic molecules, peptides, peptide mimetics, polypeptides, nucleic acid ligands, aptamers, and antibodies that bind to a SDF-1 polypeptide and reduce its biological activity. Methods of assaying SDF-1 biological activity include monitoring tumor growth rate, tumor size, neovasculogenesis, bone marrow-derived contribution to tumor neovessels, or otherwise monitoring perfusion of a neoplastic tissue.

In one particular example, a candidate compound that binds to an SDF-1 polypeptide may be identified using a chromatography-based technique. For example, a recombinant SDF-1 polypeptide of the invention may be purified by standard techniques from cells engineered to express the polypeptide, or may be chemically synthesized, once purified the peptide is immobilized on a column. A solution of candidate agents is then passed through the column, and an agent that specifically binds the SDF-1 polypeptide or a fragment thereof is identified on the basis of its ability to bind to SDF-1 polypeptide and to be immobilized on the column. To isolate the agent, the column is washed to remove non-specifically bound molecules, and the agent of interest is then released from the column and collected. Agents isolated by this method (or any other appropriate method) may, if desired, be further purified (e.g., by high performance liquid chromatography). In addition, these candidate agents may be tested for their ability to modulate vasculogenesis, (e.g., as described herein). Agents isolated by this approach may also be used, for example, as therapeutics to treat or prevent the onset of a disease or disorder characterized by undesirable vasculogenesis, or to treat or prevent a neoplasia (e.g., lung cancer, melanoma, pancreatic cancer, lymphoma, leukemia). Compounds that are identified as binding to a SDF-1 polypeptide with an affinity constant less than or equal to 1 nM, 5 nM, 10 nM, 100 nM, 1 mM or 10 mM are considered particularly useful in the invention.

Such agents may be used, for example, as a therapeutic to combat a neoplasia or to prevent the relapse of a neoplasia following remission of a neoplastic disease. Optionally, agents identified in any of the above-described assays may be confirmed as useful in conferring protection against the development of a neoplasia in any standard animal model (e.g., tumor growth in a rodent model, such as a rodent injected with a neoplastic cell).

Each of the polynucleotide sequences provided herein may also be used in the discovery and development of antineoplastic compounds (e.g., chemotherapeutics, therapeutic antibodies). The SDF-1 protein, upon expression, can be used as a target for the screening of agents that bind SDF-1 and reduce its biological activity. The SDF-1 antagonists of the invention may be employed, for instance, to inhibit and treat a variety of neoplasias, including but not limited to lung cancer, melanoma, pancreatic cancer, lymphoma, leukemia.

### Test Compounds and Extracts

In general, SDF-1 antagonists (e.g., agents that specifically bind and inhibit the activity of a SDF-1 polypeptide) are identified from large libraries of natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Agents used in screens may include known those known as therapeutics for the treatment of neoplasias. Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

Libraries of natural polypeptides in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Numerous methods are also available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to have SDF-1 binding and/or inhibitory activity further fractionation of the positive lead extract is necessary to isolate molecular constituents responsible for the observed effect. Thus, the goal of the extraction, fractionation, and purification process is the careful characterization and identification of a chemical entity within the crude extract that reduces tumor growth rate, tumor size, neovasculogenesis, or bone marrow-derived contribution to tumor neovessels in a cell. Methods of fractionation and purification of such heterogenous extracts are known in the art. If desired, compounds shown to be useful as therapeutics are chemically modified according to methods known in the art.

### Administration of Compositions

The invention provides a simple means for identifying compositions (including nucleic acids, peptides, small molecule inhibitors, aptamers, and antibodies) capable of binding to and inhibiting the activity of SDF-1. Such agents are useful as therapeutics for the treatment or prevention of a neoplasia. Accordingly, a chemical entity discovered to have medicinal value using the methods described herein is useful as a drug or as information for structural modification of existing compounds, e.g., by rational drug design. Such methods are useful for screening agents having an effect on a variety of conditions characterized by a reduction in innate immunity.

The compositions described above may be administered to animals including rodents and human beings in any suitable formulation. Compositions of the invention may be administered to the subject neat or in pharmaceutically acceptable carriers (e.g., physiological saline) in a manner selected on the basis of mode and route of administration and standard pharmaceutical practice. Preferable routes of administration include, for example, subcutaneous, intravenous, interperitoneally, intramuscular, or intradermal injections that provide continuous, sustained levels of the drug in the patient. Compositions for modulating angiogenesis may be formulated in pharmaceutically acceptable carriers or diluents such as physiological saline or a buffered salt solution. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

### Administering Viral Vectors to a Subject

Viral vectors can be used in a method for modulating angiogenesis in a subject. In this method, a viral vector (e.g., AAV) having a nucleic acid encoding VEGF is administered to an animal in a manner in which the nucleic acid becomes expressed. Administration of viral vectors (e.g., AAV) to an animal can be achieved by direct introduction into the animal, e.g., by intravenous injection, intraperitoneal injection, or in situ injection into target tissue. For example, a conventional syringe and needle can be used to inject a viral vector particle suspension into an animal. Depending on the desired route of administration, injection can be in situ (i.e., to a particular tissue or location on a tissue), intramuscular, intravenous, intraperitoneal, or by another parenteral route.

Parenteral administration of vectors or vector particles by injection can be performed, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the vectors or vector particles may be in powder form (e.g., lyophilized) for constitution with a suitable vehicle, for example, sterile pyrogen-free water, before use.

### Effective Doses

The compositions described above are preferably administered to a mammal (e.g., rodent, human) in an effective amount, that is, an amount capable of producing a desirable result in a treated subject (e.g., modulating angiogenesis in the subject). Toxicity and therapeutic efficacy of the compositions utilized in methods of the invention can be determined by standard pharmaceutical procedures. As is well known in the medical and veterinary arts, dosage for any one animal depends on many factors, including the subject's size, body surface area, age, the particular composition to be administered, time and route of administration, general health, and other drugs being administered concurrently.

The amount of the therapeutic agent to be administered varies depending upon the manner of administration, the age and body weight of the patient, and with the clinical symptoms of the neoplasia. Generally, amounts will be in the range of those used for other agents used in the treatment of other diseases associated with neoplasia, although in certain instances lower amounts will be needed because of the increased specificity of the compound. A compound is administered at a dosage that inhibits SDF-1 biological activity, as assayed by identifying a reduction in tumor growth rate, tumor size, neovasculogenesis, or bone marrow-derived contribution to tumor neovessels in a neoplastic tissue or organ as determined by a method known to one skilled in the art, or using any that assay that measures the expression or the biological activity of a SDF-1 polypeptide. In a typical embodiment, 100 mg/kg is administered.

### Treating Neoplasia

Accordingly, the present invention provides methods of treating neoplastic diseases and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a neoplastic disease, such as leukemia, or disorder or symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which an excess of SDF-1 signalling may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with neoplasia, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

The administration of a compound for the treatment of a neoplasia may be by any suitable means that results in a concentration of the therapeutic that, combined with other components, is effective in ameliorating, reducing, or stabilizing a neoplasia. The compound may be contained in any appropriate amount in any suitable carrier substance, and is generally present in an amount of 1-95% by weight of the total weight of the composition. The composition may be provided in a dosage form that is suitable for local or systemic administration (e.g., intratumoral, parenteral, subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a neoplasia by using carriers or chemical derivatives to deliver the therapeutic agent to a particular cell type (e.g., a neoplastic cell, or bone marrow-derived endothelial cell precursor). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a neoplasia, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

As indicated above, the pharmaceutical compositions according to the invention may be in the form suitable for sterile injection. To prepare such a composition, the suitable active therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

Controlled release parenteral compositions may be in form of aqueous suspensions, microspheres, microcapsules, magnetic microspheres, oil solutions, oil suspensions, or emulsions. Alternatively, the active agent may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the active anti-neoplasia therapeutic substance). The coating may be applied on the solid dosage form in a similar manner as that described in Encyclopedia of Pharmaceutical Technology, supra.

At least two anti-neoplasia therapeutics (e.g., a SDF-1 specific antibody and a VEGF-specific antibody) may be mixed together in the tablet, or may be partitioned. In one example, the first active anti-neoplasia therapeutic is contained on the inside of the tablet, and the second active anti-neoplasia therapeutic is on the outside, such that a substantial portion of the second active anti-neoplasia therapeutic is released prior to the release of the first active anti-neoplasia therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment. Compositions as described herein can also be formulated for inhalation and topical applications.

### Controlled Release Oral Dosage Forms

Controlled release compositions for oral use may, e.g., be constructed to release the active anti-neoplasia therapeutic by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Combination Therapies

Optionally, an anti-neoplasia therapeutic may be administered in combination with any other standard anti-neoplasia therapy; such methods are known to the skilled artisan and described in Remington's Pharmaceutical Sciences by E. W. Martin. In particular embodiments, an effective amount of an antibody or other agent that specifically binds SDF-1 and reduces its biological activity is administered in combination with an antibody that binds to VEGF. In particular embodiments, the anti-SDF-1 antibody is administered in combination with a VEGF specific antibody, such as bevacizumab or ZD6474, vascular disrupting agents, such as combrestatin or Oxi4503, and Tie-2 inhibitors, such as AMG-386. Combinations are expected to be advantageously synergistic. Therapeutic combinations that decrease tumor perfusion, vascular volume, microvascular density, or the number of viable, circulating endothelial and progenitor cells, are identified as useful in the methods of the invention.

### Kits

The invention provides kits for the treatment or prevention of neoplastic disease or diseases characterized by an undesirable increase in vasculogenesis. In one embodiment, the kit includes a therapeutic or prophylactic composition containing an effective amount of an agent that specifically binds an SDF-1 polypeptide, such as an SDF-1 specific antibody, in unit dosage form. If desired, the kit also contains an effective amount of a VEGF antibody, such as Bevacizumab. Kits could contain other combinations such as antibodies to SDF-1 plus vascular disrupting agent and/or Tie-2 inhibitor. In some embodiments, the kit includes a sterile container which contains a therapeutic or prophylactic cellular composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, a cell of the invention is provided together with instructions for administering the agent to a subject having or at risk of developing a neoplastic disease. The instructions will generally include information about the use of the composition for the treatment or prevention of a neoplastic disease. In other embodiments, the instructions include at least one of the following: description of the therapeutic agent; dosage schedule and administration for treatment or prevention of neoplasia or symptoms thereof, precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### Compositions and Methods for Treatment

A method of treating leukemia or preventing a leukemia relapse in a subject in need thereof includes administering to the subject an agent that binds to SDF-1 and reduces SDF-1 biological activity (e.g., antibody), thereby treating leukemia or preventing leukemia relapse in the subject. Generally, about 0.05-200 mg/kg of the SDF-1 specific antibody is administered (e.g., 1 mg/kg, 5 mg/kg, 8 mg/kg, 20 mg/kg, 30 mg/kg, 50 mg/kg, 70 mg/kg, 90 mg/kg, 100 mg/kg, 120 mg/kg, 140 mg/kg, 160 mg/kg, 180 mg/kg, 200 mg/kg). In one embodiment, about 1-100 mg/kg is administered. A pharmaceutical composition for the treatment of leukemia or the prevention of a leukemia relapse includes an effective amount of an SDF-1 antibody that specifically binds SDF-1 and blocks SDF-1 biological activity. The composition can further include an agent that inhibits VEGF biological activity (e.g., bevacizumab).

A therapeutic regimen for reducing marrow mobilization in a subject following chemotherapy for the treatment of a neoplasia includes administering conventional chemotherapy to the subject, and subsequently administering an agent that binds to SDF-1 and reduces SDF-1 biological activity. The agent can be administered between about 7-60 days (e.g., between about 10 to 45 days, between about 14 to 28 days, etc.) following chemotherapy. In one embodiment, the agent is administered while marrow endothelial progenitor cells are mobilized. In order to determine when marrow endothelial cells are mobilized in a subject, one can obtain blood from the subject, and look for circulating endothelial cells, endothelial progenitor cells, and/or vascular precursor cells. The agent can be administered (e.g., intravascularly) in combination with a vascular disrupting agent and/or an agent that reduces VEGFR2 biological activity.

The invention provides for a method for the treatment of a primary refractory neoplasia (e.g, a neoplasia not sensitive to treatment; a cancer that grows during therapy; hemotalogic disease complete remission) in a subject. In one example, the method includes identifying a subject as having a primary refractory neoplasia (e.g, during or after treatment cycle, continued cancer growth is measured by radiologic imaging, blood lab testing, physical exam, etc.), and administering an agent that binds SDF-1α and reduces SDF-1 biological activity, thereby treating the primary refractory neoplasia. A primary refractory neoplasia can be leukemia, for example. The method can further include administering an agent that inhibits VEGF biological activity (e.g., bevacizumab). In some embodiments, the agent that binds SDF-1 and the bevacizumab are delivered intravascularly. Administering can be during marrow progenitor cell mobilization, and/or between about fourteen and twenty-eight days after cytotoxic chemotherapy.

A method of treating or preventing intimal hyperplasia includes administering an agent that binds SDF-1α and reduces SDF-1 biological activity, thereby treating or preventing intimal hyperplasia. A method of preventing recruitment of an endothelial progenitor cell to a site of ischemic injury includes administering to the eye an agent that binds SDF-1α and reduces SDF-1 biological activity (e.g., an anti-SDF-1a antibody), thereby preventing recruitment of an endothelial progenitor cell to the site. The method can be used to treat or prevent vascular retinopathy or hemangioma. A cellular composition includes at least about 50% bone marrow-derived CD133⁺CXCR4⁺ that function in vessel formation. A method for identifying a myelomonocytic endothelial-like cell having proangiogenic potential includes identifying a bone marrow derived CD133⁺CXCR4⁺ cell.

A method for treating tissue ischemia includes administering to a patient in need thereof a cellular composition that includes at least about 50% bone marrow-derived CD133⁺CXCR4⁺ that function in vessel formation, thereby treating the tissue ischemia. The tissue ischemia can be myocardial ischemia, limb ischemia, thrombotic stroke, or organ ischemia.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Materials and Methods

Generation of gfp chimeric mice: The gfp transgenic mouse strain used as the donor strain was obtained from The Jackson Laboratory (Bar Harbor, Me.). The strain carries gfp driven by chicken beta-actin promoter and CMV immediate early enhancer. All cell types within this animal express gfp. C57B6.gfp radiation chimeric mice (n=46) were generated by irradiating recipient C57BL6 mice with 950 rads followed by intravenous injection of either whole BM (1×10⁶) or purified hemangioblasts (2×10⁵) from gfp⁺ donor mice. Hemangioblasts were purified from adult BM as follows: harvested marrow was made into a single cell suspension and plated onto treated plastic dishes in IMDM+20% FBS for 4 hrs. Non-adherent cells were collected and three rounds of lineage antibody depletion (B220, CD3, CD4, CD8, CD11b, Gr-1, TER 119) were performed with the Miltyni magnetic activated cell sorting (MACS) system until a small aliquot stained with PE-conjugated Lineage Ab cocktail showed >95% lineage negative by FACS. The Lin⁻ cells were then positively selected for Sca-1 for 2-3 rounds until an aliquot showed greater then 95% Sca-1⁺, Lin⁻ purity had been achieved. The Sca-1⁺, Lin⁻ cells were then stained for CD45 to confirm hematopoietic origin. For the serial transplants approximately 1,000 re-purified hemangioblasts were transplanted. For single hemangioblast transplants Sca-1+, c-kit+, Lin− hemangioblasts were enriched by FACS sorting prior to individual hemangioblast selection with micromanipulators via fluorescent microscopy. Individual Gfp+ hemangioblasts were then mixed with 2×10⁵ non-Gfp+BM cells that had been depleted of Sca-1+ cells by magnetic beads prior to transplant into irradiated hosts.

Induction of retinal neovascularization: After durable hematopoietic reconstitution was established, chimeric mice were injected i.o. with AAV-VEGF followed at one month with i.p. 10% sodium fluorescein. Fifteen minutes later, they underwent laser treatment. An Argon Green laser system (HGM Corporation, Salt Lake City, Utah) was used for retinal vessel photocoagulation with the aid of a 78-diopter lens. The blue-green argon laser (wavelength 488-514 nm) was applied to selected venous sites next to the optic nerve. Venous occlusion was accomplished using laser parameters of 1-sec duration, 50 μm spot size, and 50-100 mW intensity.

Data collection and analysis: Three weeks after laser treatment, mice were killed and their eyes enucleated. Technical limitations prevented the use of flat-mounted retinas for both confocal microscopy and for immunocytochemistry. The thickness of the retina (approximately 200 microns) prevented adequate penetration of antibody. Therefore, selected mice (n=10) were perfused with buffer containing Hoechst stain in order to label nuclei and delineate the vascular lumen. Eyes (n=20) from treated radiation chimeras were sectioned and stained with hematoxylin. Sections were counter-stained with PE-conjugated anti-Factor VIII or Biotin-conjugated anti-PECAM-1 and anti-MECA-32 followed by avidin-PE (BD BioSciences, San Jose, Calif.) to identify endothelial cells. A minimum of 30 sections per eye was examined for the presence of gfp⁺, PE⁺ cells.

This methodology prevented the visualization of intact capillary tufts detectable by confocal microscopy of whole flat-mounted retina. For confocal visualization mice (n=36) were perfused with 3-5 mL of 50 mg/mL tetramethyl rhodamine isothiocyanate (TRITC)-conjugated dextran (160,000 avg. MW, Sigma Chemical Co., St. Louis, Mo.) in phosphate-buffered formaldehyde, pH 7.4, administered through the left ventricle. Immediately afterwards, the eyes were removed and the retinas dissected and mounted flat for confocal microscopy using Vectashield mounting medium (Vector Laboratories, Burlingame, Calif.) to inhibit photo-bleaching. The Olympus IX-70, with inverted stage, attached to the Bio-Rad Confocal 1024 ES system was used for fluorescence microscopy. A Krypton-Argon laser with emission detector wavelengths of 598 nm and 522 nm was used to differentiate the red and green fluorescence. The lenses used on this system were the (Olympus) 10×/0.4 Uplan Apo, 20×/0.4 LC Plan Apo, 40×/0.85 Uplan Apo, 60×/1.40 oil Plan Apo and 100×/1.35 oil Uplan Apo. The software was OS/2 Laser Sharp. Peripheral blood and BM was also collected for donor contribution analysis by FACS with lineage specific antibodies conjugated to PE (BD BioSciences, San Jose, Calif.).

### Example 2

### Transplanted Animals Exhibit Functional Hemangioblast Activity

Irradiated C57BL6 recipients were transplanted with either whole BM, or highly enriched hemangioblasts, or single hemangioblast from ubiquitous gfp mouse donors to generate radiation chimeras that are designated as C57BL6.gfp. A FACS analysis of purified hemangioblasts and multilineage reconstitution was performed. Transplanted cells were deemed to be highly enriched for hemangioblasts as a result of 1) selection by differential adherence (MSC adhere to tissue culture plastic, hemangioblasts do not), and 2) further selection of non-adherent cells by Ficoll centrifugation followed by purification for Sca-1⁺ Lin⁻ phenotype. Single gfp⁺ hemangioblasts were initially purified as a Sca-1⁺, c-kit⁺, Lin⁻ population by FACS followed by individual selection with micromanipulators via fluorescent microscopy prior to transplant. In these chimeras, marrow- or hemangioblast-derived cells express gfp were identified visually as green cells. A combination of site-specific growth factor over-expression and ischemic insult was used to elicit retinal neovascularization in these C57BL6.gfp chimeras. Initial studies attempted to use either laser occlusion or local expression of VEGF, but either treatment alone failed to result in consistent neovascularization. In contrast, the combination of site-specific VEGF expression followed by laser-induced venous occlusion resulted in increased numbers of preretinal vessels and consistent preretinal neovascularization.

Initially, primary hemangioblast transplant recipients were monitored for durable, multilineage hematopoietic reconstitution prior to ischemic induction. Six months post hemangioblast transplant and one month after injury, the eyes from treated radiation chimeras were sectioned and stained with hematoxylin. Animals were perfused with buffer containing Hoechst stain in order to label nuclei and delineate the vascular lumen. Induction of a pre-retinal neovascularization was observed in a mouse eye that underwent intravitreal injection with recombinant AAV containing the VEGF gene followed by laser-induced venous occlusion. A gfp⁺, hemangioblast-derived green endothelial cell has integrated into the lumen of a vessel. The sections were counter-stained with either PE-conjugated anti-Factor VIII, or anti-PECAM-1 and anti-MECA-32 conjugated to biotin with a PE-avidin secondary to confirm the endothelial nature of the green cell. It was clearly observed that the gfp⁺ cells that surround the lumens (outlined by Hoechst staining) also react with the red fluorescent Factor VIII or PECAM-1 antibodies to produce yellow cells in the combined images, thus, confirming that they are endothelial cells of donor origin. Examination of a minimum of 30 sections per retina, sampling on both sides of the optic nerve, demonstrated that every gfp⁺ cell found surrounding a lumen reacted with an endothelial cell specific antibody. The pattern of vascular development induced in the model was readily seen in flat mounts of retina perfused with red fluorescent-labeled dextran. In fluorescence micrographs, the patterns of vascular development in C57B6.gfp chimeric mice that underwent intravitreal injection with AAV followed by laser-induced venous occlusion were detected. A representative flat mount of the entire retina of an injured ischemic eye showed areas with newly regenerated blood vessels that have endothelial cells derived from donor gfp⁺ hemangioblasts. The non-injured eye of the same animal showed gfp⁺ cells circulating through intact vessels with no apparent contribution to endothelial cells of the vessels. In injured eyes, cells expressing gfp were seen throughout the retinal vasculature, including within capillary tufts that represent new vascular growth. Numerous areas of newly formed capillary tubes expressing gfp were observed that stretch across areas of preexisting vasculature. Merged green and red channels visualized the yellow tubes of newly formed blood vessels because gfp⁺ endothelial cells have produced lumens capable of being perfused with rhodamine. Importantly, all five mice transplanted with highly enriched hemangioblasts (˜95% Sca-1⁺, Lin⁻) contained numerous new vessels composed of gfp⁺ cells, which were easily visualized in all quadrants of the retina. A minimum of two new areas of neovascularization were observed in every field examined, indicating most injuries were repaired, in part, with donor derived cells. In contrast, mice (n=10) receiving whole BM exhibited a decreased frequency of gfp⁺ cells in areas of neovascularization. This indicates that the greater the number of hemangioblasts transplanted to generate the chimera the greater the number of donor-derived endothelial cells that can be visualized. All transplanted animals, however, exhibited functional hemangioblast activity, as defined by repopulation of the blood and regeneration of blood vessels that co-enriches with the hemangioblast.

The classic definitive assay for HSC function in murine models is durable long-term reconstitution of the hematopoietic system in irradiated hosts. Subsequent transplantation of secondary lethally irradiated hosts with enriched BM hemangioblasts from primary transplants demonstrated the ability of HSC to expand and self-renew, thus satisfying the definition of a stem cell. Serial transplantation tends to preclude MSC participation in the reconstitution because there is no evidence to indicate that MSC are able to serially engraft. To confirm that the production of “green” endothelial cells observed during neovascularization was a property of the self-renewing long-term repopulating HSC, secondary transplants of highly purified HSC derived from primary recipients were performed. For primary recipients, five animals were lethally irradiated and transplanted with highly purified gfp⁺ HSC. Animals were monitored for 10 months to ensure durable, multilineage hematopoietic reconstitution. Gfp⁺ HSC were then purified from the primary recipients and transplanted into five secondary hosts for induction of hemangioblast activity. Following confirmation of multilineage hematopoietic reconstitution in the secondary recipients after three months, these animals were scored for hemangioblast activity derived from gfp⁺ HSC. Retinal ischemia was induced and resultant neovascularization was analyzed at four months post secondary transplant. The vascular lumens from a treated retina perfused with rhodamine indicated that gfp⁺ endothelial cells have participated in the regeneration of induced capillaries. The combined image shows that donor HSC-derived endothelial cells regenerated the entire vascular tuft in the secondary transplant recipient. Thus, a serially transplantable, multiple hematopoietic lineage reconstituting adult HSC (i.e., hemangioblast) clearly has hemangioblast properties and can regenerate functional vasculature.

In order to determine if a single HSC clone could make both blood and blood vessels, the model was repeated with animals that were reconstituted with a single HSC. Following FACS sorting, individual Gfp+, Sca-1+, c-kit+, Lin− HSC were manually isolated and transplanted along with 2×10⁵ Sca-1 depleted non-Gfp marrow. The depleted marrow served as a source of short-term hematopoietic progenitors to enhance single HSC engraftment. The single HSC provided multilineage hematopoietic reconstitution and robust endothelial cell contributions to new vessel formation. This new vessel formation was observed in all three animals undergoing single HSC transplantation and provides definitive proof that a single adult HSC can function as a hemangioblast.

### Example 3

### Inhibition of Retinal Neovascularization

The hemangioblast model described above in Examples 1 and 2 was used to test the effect of anti-SDF-1 antibody on retinal neovascularization. In the present example, the hemangioblast model featuring a particular modification was used. Specifically, the eyes of the mice were injected with antibody that blocked SDF-1 activity. This experiment showed that retinal neovascularization was blocked by neutralizing SDF-1 activity (e.g., intravitreal injection of anti-SDF-1 antibodies). Treatment of the eye completely blocked gfp+ hemangioblast-derived neovascularization of ischemic retinas. Fluorescence confocal micrographs showed blocking of hemangioblast-driven neovascularization by anti-SDF-1 antibody. Normal incorporation of gfp+ hemangioblast derived cells into new blood vessels in the rodent eye was observed. The blocking of SDF-1 activity in the eye prevented the incorporation of Gfp+ hemangioblasts into blood vessels. An example of a protocol for blocking Gfp+ hemangioblast-derived neovascularization of ischemic retinas is detailed below.

First, GFP males were euthanized by cervical dislocation under general anesthesia and BM from the males was harvested. Cells stained for c-kit APC and sca-1 PE were FACS sorted. While cells were sorting, BL6 females were lethally irradiated (850 RADS). Next, a BM transplantation was performed by retinal orbital sinus (ROS) injections with the sorted cells on the irradiated BL6 mice. Three weeks after the ROS injections, tail bleeds were performed. The FACS Caliber was used to check for engraftment of the sca-1/c-kit cells. Next, recombinant AAV (rAAV)-VEGF was injected intravitreally into the right eye of the positively engrafted mice. Four weeks after the VEGF injections, retinal laser photocoagulation was performed in the right eye. Immediately following the lasering procedure, monoclonal anti-SDF-1 antibody (R&D Systems MAB310) was injected intravitreally in the right eye. PBS was intravitreally injected into the untreated eye. Intravitreal injection of anti-SDF-1 antibody was performed once every week for the following four weeks. Animals were then anesthetized and perfused by cardiac puncture (left ventricle) with 3 ml TRITC-Dextran in 4% buffered formaldehyde. The retinas from both treated and untreated eyes were subsequently dissected. The retinas were mounted flat in buffered glycerin and imaged by confocal microscopy.

Using additional cohorts of animals, the experiment described above was repeated. Animals were treated with either SDF-1 antibody or mock-treated with PBS via intravitreous injections, and then retinal ischemia was induced in the animals. The animals were perfused with red dye to show vessels and imaged by confocal microscopy. The SDF-1 antibody injections completely blocked gfp+ hemangioblast derived green blood vessel formation while the mock injected animals formed green gfp+ hemangioblast derived blood vessels as expected in response to retinal ischemia. Fluorescence confocal micrographs of multiple mice transplanted with either gfp+ hemangioblasts or mock-injected with PBS showed vessels that formed subsequent to induction of ischemia. Gfp+ hemangioblast derived green blood vessels showed normal function. Confocal imaging showed that new blood vessel formation in animals that received intravitreous injection of anti-SDF-1 antibody did not occur.

### Example 4

### Marrow Contributes to Blood Vessels in Cancers

Irradiated C57BL/6 recipients were transplanted with whole bone marrow from mouse donors ubiquitously expressing green fluorescent protein (GFP). Transplant recipients (n=40) were monitored for durable, multilineage hematopoietic reconstitution. Six months after transplantation mice were inoculated with lung cancer cells (LLC), pancreatic cancer cells (PAN02), melanoma cells (B16) and lymphoma cells (EL4). Tumors were harvested when they measured between 500 mm³ and 600 mm³ in volume. Typically, lung cancers took 14 days to achieve this volume; pancreatic cancer took 16 days to reach this volume; melanoma took 20 days to reach this volume; and lymphoma took 14 days to reach this volume. To assess whether bone marrow-derived cells were involved in tumor neovascularization, immunohistochemistry and confocal microscopy was performed for endothelial cell surface proteins. Marrow-derived cells (GFP⁺) co-expressing hematopoietic surface proteins CD45, CD11b and CD14 were identified. These cells were found in pockets away from tumor blood vessels. Tumor sections were also analyzed for cells co-expressing GFP and endothelial cell surface proteins, platelet endothelial cell adhesion molecule 1 (PECAM-1, CD31) and vonWillibrand's factor (vWF). All recipient mice with GFP hematopoietic reconstitution demonstrated marrow contribution of endothelial-like cells lining blood vessels lumens in cancers. Blood vessels containing at least one marrow-derived (GFP) endothelial cell (coexpressing CD31 and vWF) were identified. These vessels represented approximately 25% of total blood vessels in lung cancers, 0.2% in pancreatic cancers, 1.5% in melanomas, and 0.1% in lymphomas. Within each animal, the degree of donor-derived cancer neovessel incorporation was directly proportional to the level of donor-derived hematopoietic engraftment. Furthermore, the majority of donor-derived vasculogenesis occurred in the periphery of the tumor.

Confocal microscopy with 0.5 micron Z-step analysis was used to identify nucleated cells (DAPI blue) co-expressing donor GFP (green) and endothelial proteins (CD31 or vWF, red). Step-wise Z-stack analysis delineated individual cells lining the tumor vasculature. This high-resolution analysis technique also constructed orthogonal views of all cells lining blood vessels. Vertical and horizontal planes permitted right-angle perspectives to critically scrutinize whether endothelial cell surface protein staining (CD31, red) co-localized with the endothelial surface of GFP donor-derived marrow cells (blue nuclei, green cytoplasm). These experiments showed that bone marrow and the HSC contribute to lung cancer endothelium.

If donor marrow-derived endothelialization of tumor vasculature was due to fusion between HSC/HPC and resident tumor vasculature then cells lining the tumor vasculature would be expected to express surface proteins of hematopoietic developmental fate. This was not observed. Instead, tumor infiltrating leukocytes (CD45) of donor origin were detected within inner pockets of tumor. None of the marrow-derived endothelial cells demonstrated co-localization of CD45.

### Example 5

### Tumor Blood Vessel Cells Derived from a Clonal Self-Renewing HSC

In order to define which cell type in bone marrow is responsible for contributing tumor neovessels, the potential of hematopoietic stem cells (HSC) was examined. The adult HSC has been shown to be capable of repairing blood vessels in ischemic retinas (Grant et al., Nat. Med. 2002; 8:607-612; Cogle et al., Blood. 2004; 103:133-135). Ischemic environments also exist within cancer, triggering hypoxia inducible factors (Vaupel et al., Cancer Res. 1989; 49:6449-6465). This finding lead to the hypothesis that HSC could provide hemangioblast activity in cancer neovascularization. The classic definitive assay for HSC function in murine models is single cell transplant to demonstrate clonality or durable, multi-lineage reconstitution after serial transplantations to demonstrate self-renewal. Both assays were combined (secondary transplantation from single HSC transplant donors) in order to rigorously test hemangioblast activity of the self-renewing and clonal HSC.

Irradiated C57BL/6 recipients were transplanted with single HSC from mouse donors ubiquitously expressing GFP. Single GFP HSCs were initially enriched as a Sca-1⁺ c-kit⁺ (SK) population by FACS followed by individual selection with micromanipulators prior to transplant. Transplant recipients were monitored for durable, multilineage hematopoietic reconstitution before serial re-transplant. Out of 80 single HSC transplant recipients, three demonstrated longterm, multilineage GFP hematopoietic chimerism. Bone marrow from the three engrafted mice was then isolated and serially transplanted into twenty secondary recipients. Durable, multilineage engraftment was established in nine out of twenty mice serially transplanted with a single HSC. Six months after secondary transplantation from single HSC donors, mice were inoculated with lung cancer cells (LLC). The kinetics of tumor growth did not differ from that of primary mice transplanted with whole bone marrow. Tumors were palpable 10 days after inoculation and were harvested at day 14. These tumors typically measured 500 mm³ to 600 mm³ in volume. At time of euthanasia and tumor collection, an evaluation of hematopoiesis was performed to verify long-term, multilineage reconstitution. In the 9 reconstituted mice, 50% of leukocytes were GFP positive. Donor GFP hematopoiesis generated multi-lineage hematopoiesis (14% monocytes/macrophages, 11% B lymphocytes, 5% T lymphocytes). Spectral confocal microscopy was used to evaluate tumors in secondary recipients of single HSC transplant. Lung cancers in all mice (n=9) demonstrated donor HSC-derived cells. Approximately 5% of tumor vasculature contained donor (green) endothelial cells lining blood vessel lumens. The degree of donor-derived endothelialization was directly proportional to the level of donor-derived hematopoietic engraftment. Specifically, tumor sections were analyzed for cells co-expressing GFP and the endothelial surface proteins CD31 and vWF. Donor-derived leukocytes (CD45) were present in the tumor; however, such cells were not found lining the vessel walls. Blood vessels were predominately located in the periphery of the tumor. These results show that adult HSC contributed to cancer neovessels.

### Example 6

### Factors Involved in Leukocyte Trafficking Affect Marrow Contribution to Cancer Blood Vessels

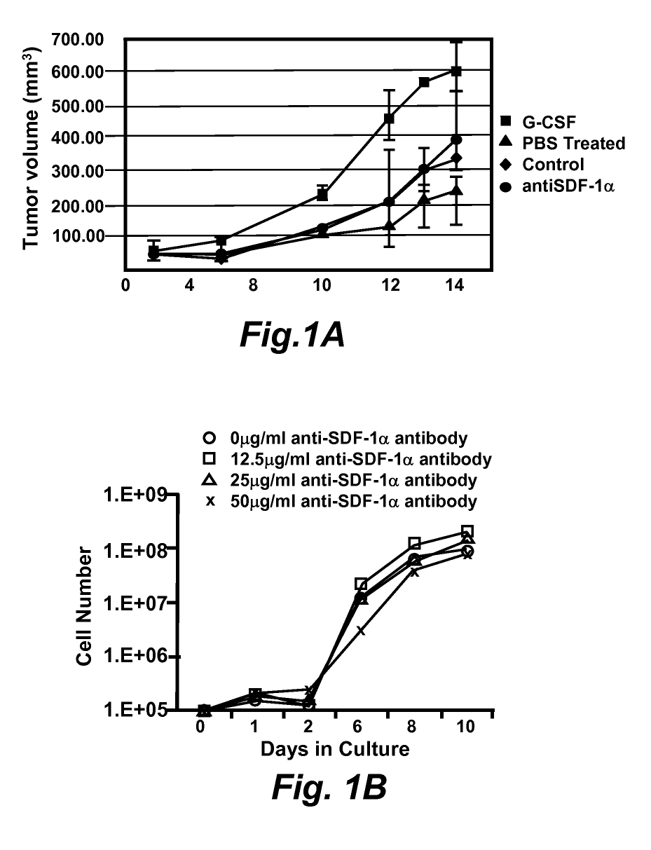

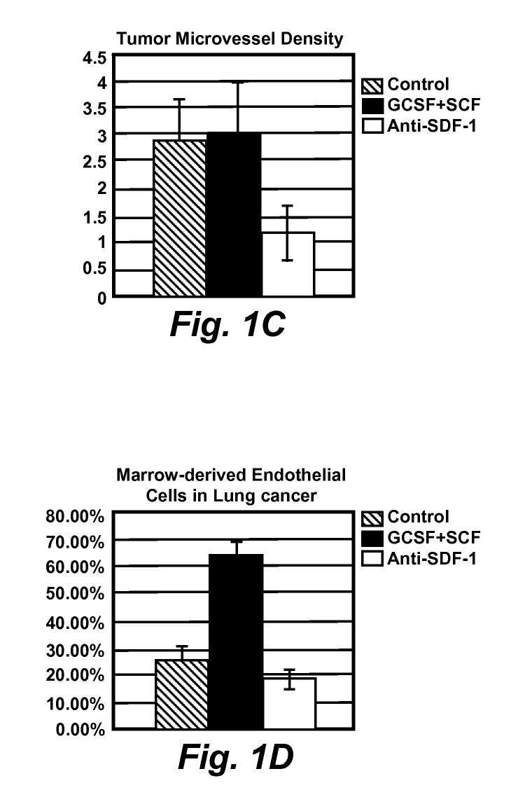

HSC can produce both blood and blood vessels in cancer. To determine whether factors involved in leukocyte trafficking are important to regulating the contribution to cancer blood vessels, slight modifications were made to the tumor model. After GFP transplant and lung cancer inoculation, cytokines involved in marrow cell mobilization (i.e., granulocyte colony stimulating factor (G-CSF) and stem cell factor (SCF) were administered. One cohort of mice (n=8) received daily subcutaneous G-CSF injections and every third day SCF was administered intravenously for 14 days. At the end of 14 days, all mice demonstrated elevated white blood counts. One control cohort of mice received no injections (n=4). Another control cohort received intratumoral PBS injections (n=4). Under these conditions, lung cancer growth in all animals was measured daily. Over 14 days, the tumors in the G-CSF and SCF treated groups grew at a faster rate and to a larger size than tumors in the control mice (FIG. 1A). In cytokine treated animals, microvessel density was not different compared to control mice (FIG. 1I); however, marrow-derived cells in the wall of tumor blood vessels was markedly elevated in the cytokine treated group compared to control mice (63% vs. 26%) (FIG. 1J). SDF-1/CXCR4 axis plays a pivotal role in marrow cell homing and migration. To determine whether blocking this axis would block marrow-derived blood vessels in cancer, GFP marrow was transplanted into wild-type C57BL/6 mice and then these mice were inoculated with lung cancer (n=8). Mice were injected intratumorally with anti-SDF-1 antibodies every day for 14 days. The same control cohorts (n=16) were used as in the cytokine treated experiment. Over the ensuing 14 days, tumors in the anti-SDF-1 treated group grew at a much slower rate and to a much smaller size, if at all (FIG. 1A). Microvessel density was markedly decreased in the anti-SDF-1 cohort compared to the control cohort (FIG. 1I). Moreover, marrow contribution to tumor neovessels was decreased in the anti-SDF-1 treated tumors compared to controls (FIG. 1J). Without wishing to be tied to theory, it is possible that the anti-neoplastic effect of anti-SDF-1 antibodies was due to a direct cytotoxic effect of the antibodies on growing cancer cells. To test this possibility, in vitro cultures of LLC lung cancer cells were established in the presence of escalating concentrations of anti-SDF-1 antibodies. Concentrations equal to and above the levels used in the in vivo animal experiments were used. In vitro cell growth was similar between all cultures, regardless of the presence of anti-SDF-1 and regardless of antibody concentration. These results indicate that a direct cytotoxic effect is unlikely to cause tumor inhibition. Again, without wishing to be tied to theory, anti-SDF-1 antibody treatment may impair neovasculogenesis and underlie the cancer inhibition observed during anti-SDF-1 treatment.

Based on the observation that marrow- and HSC-derived vasculogenesis occurs in the setting of physiologic repair (Slayton et al., Stem Cells. 2007; 25:2945-2955; Grant et al., Nat. Med. 2002; 8:607-612; Cogle et al., Blood. 2004; 103:133-135), it appeared likely that bone marrow and HSC exhibited hemangioblast activity in the pathologic setting of tumor neovascularization. Using a transplant model with tumor implantation, results reported herein clearly demonstrate a bone marrow contribution to blood vessels in cancers of the lung, pancreas, skin, and lymphatics. In this study, marrow-derived cells contributing to cancer blood vessels exhibited three features of endothelial cells: (1) cell surface expression of two typical proteins (CD31 and vWF), (2) luminal orientation, and (3) lack of hematopoietic surface proteins. These cells looked like endothelial cells and acted like endothelial cells, but given the in vitro possibility that monocytes and macrophages also perform these activities, these in vivo identified cells should more appropriately be classified as tumor endothelial scar cells.

These studies provide a straightforward transplant model with syngeneic cancer injections and confocal microscopy to confirm tumor endothelial scar formation, which was observed in several cancers including a hematologic malignancy. Although, it should be pointed out that marrow contributions to blood vessels varied depending on the cancer. Lung cancers demonstrated more marrow-derived tumor endothelial scar cells, followed by melanoma, then pancreatic cancer or lymphoma.

To determine whether the HSC is an origin of tumor endothelial scar cells, a combined single and secondary transplant model was used. A single cell transplantation model (Grant et al., Nat. Med. 2002; 8:607-612) was used to determine whether the adult HSC provided hemangioblast activity in the setting of cancer neovessel formation. The adapted model was intended to address the limitations of previous studies and rigorously test the HSC as a clonal source of endothelial scar cells in tumor neovascularization. Results reported herein demonstrated that a clonal, self-renewing HSC was capable of generating tumor endothelia. Moreover, the level of contribution was commensurate with hematopoietic engraftment.

It seemed likely that factors involved in leukocyte trafficking also affect marrow contribution to cancer blood vessels. Specifically, cytokines mobilizing leukocytes (i.e., G-CSF and SCF) were likely to increase bone marrow-derived tumor endothelial scar cells. Similarly, blocking chemoattractants (i.e., SDF-1) was expected to decrease this activity. Results reported herein demonstrate that mobilizing leukocytes with G-CSF and SCF led to increased marrow cells in tumor blood vessels, and blocking the potent chemokine, SDF-1, inhibited tumor growth rate, size, neovasculogenesis, and the number of marrow-derived endothelial scar cells. The intensified contribution of bone marrow to cancer blood vessels after bone marrow mobilization is likely to be clinically significant.

Chemotherapy is routinely administered to treat cancers. Chemotherapy not only kills tumor cells, it also mobilizes bone marrow cells. Bursts of circulating endothelial progenitor cells (EPCs) are mobilized from the bone marrow following treatment with chemotherapy (Harris et al., 2006 Invest Ophthalmol Vis Sci 47:2108-2113). A surge of circulating EPCs between cycles of chemotherapy may replace damaged endothelial cells. Moreover, growth factors such as G-CSF are often administered after chemotherapy to hasten hematopoietic recovery. Administration of growth factors permits higher chemotherapy dose density and has supportive care uses, but it also mobilizes bone marrow cells into circulation. The findings reported herein indicate that bone marrow mobilization increased tumor neovessels and growth. This suggests that current clinical practices may have a counterproductive aspect in that they support bone marrow-derived cancer neovasculogenesis after chemotherapy.

Targeting bone marrow cell homing and migration to sites of cancer neovasculogenesis represents a therapeutic opportunity. Recently, investigators induced mobilization of bone marrow-derived cancer blood vessels after administration of a vascular disrupting agent (VDA) (Shaked et al., Science. 2006; 313:1785-1787). By additionally targeting marrow-derived hemangioblast activity with anti-VEGFR2 antibodies, tumor neovessels and tumor growth was significantly inhibited. A combination of VDA and anti-angiogenic agents lead to more effective anti-cancer therapy (Siemann et al., Int J Radiat Oncol Biol Phys. 2004; 60: 1233-1240). Clinical anti-cancer regimens including adjuvant VDAs and anti-VEGFR2 antibodies are likely to be useful for chemotherapy. Results from the studies reported herein suggest that altering the timing of anti-vascular therapy is likely to be advantageous. In particular, targeting marrow mobilization after chemotherapy (typically days 14 to 28 depending on regimen) may be advantageous Delivering anti-SDF-1 therapy in addition to anti-vascular therapy at this time of marrow mobilization may have an advantageous effect. Marrow contribution to tumors could also be exploited for early identification of metastatic disease. Since marrow-derived cells (and specifically progeny of the hematopoietic stem cell as determined by this work) contribute to tumor vasculogenesis, tracking marrow contribution to tumors may permit illumination of micrometastatic disease. For example, tagging endothelial progenitor cells, which are progeny of hematopoietic stem cells, with a radio-evident molecule such as a contrast dye would permit non-invasive imaging of metastatic cancer throughout a patient's body.

Previous reports have identified small tumors having higher levels of bone marrow-derived cancer neovessels as compared to large tumors (Nolan et al., Genes Dev. 2007; 21:1546-1558). This situation is optimal for detecting metastatic lesions, given that earlier detection of metastatic lesions may lead to better clinical outcomes. Tagging bone marrow cells and tracking them by non-invasive methods would highlight micrometastatic disease. Early detection should permit more definitive therapy. Measurement of marrow hemangioblast activity in cancer could act as a surrogate marker of anti-angiogenic activity. Choosing the optimal dose for biologic agents has many challenges compared to the pharmacodynamics of traditional cytotoxic agents. Should it be possible to utilize marrow hemangioblast activity as a surrogate marker, then optimal dosing of vascular targeting therapy could be modified based on the degree of hemangioblast activity in cancer. Finally, results reported herein indicated that targeting intratumoral SDF-1 intervenes in marrow derived contribution to cancer blood vessels. Recently, intensive research has identified the importance of the SDF-1/CXCR-4 axis in cancer growth and metastasis (Muller et al., Nature. 2001; 410:50-56; Zeelenberg et al., Cancer Res. 2003; 63:3833-3839). Tumor expression of SDF-1 is necessary and sufficient to incorporate marrow-derived cells into tumor endothelium (Aghi et al., Cancer Res. 2006; 66:9054-9064). Results reported herein indicate the importance of SDF-1 in tumor growth and suggest that anti-SDF-1 therapy on marrow contribution to cancer neovessels. The inhibition of marrow-derived hemangioblast activity in cancer by anti-SDF-1 may augment anti-vascular therapies such as VDAs and anti-VEGFR2 agents. In the future, combination therapy targeting neoplastic cells and marrow recruitment to tumor endothelium may serve as more effective anti-cancer regimens.

Results reported in Examples 4-6 were obtained using the following methods and materials.

### Murine HSC Transplant Model

All animal procedures were performed under approval of the University of Florida Animal Care and Use Committee. The C57BL/6 and GFP transgenic strains were obtained from Jackson Laboratories (Bar Harbor, Me.). Radiation chimeras were generated by irradiating recipient animals (C57BL/6 with 950 cGy) followed by intravenous transplantation of either whole GFP⁺ bone marrow (1×10⁶) or single Sca-1⁺ c-Kit⁺ (SK) GFP⁺ HSC. HSCs were purified from adult bone marrow as previously reported (Grant et al., Nat. Med. 2002; 8:607-612). Serial transplantation was performed using 950 cGy and whole bone marrow from primary recipients transplant with a single HSC. Donor hematopoietic engraftment was determined by FACS analysis of peripheral blood starting at one month post-transplant and confirmed during each subsequent procedure. Multi-lineage analysis of peripheral blood and bone marrow were performed with lineage-specific antibodies conjugated to phycoerythrin (PE, BD Biosciences). Animals that were not long-term engrafted were excluded from the study.

### Tumor Inoculation

Chimeric mice were injected with 2×10⁵ Lewis lung carcinoma cells (LLC, CRL-1642, ATCC, Manassas, Va.), 5×10⁶ pancreatic cancer cells (PAN02, ATCC), 2×10⁵ melanoma cells (B16, ATCC), and 5×10⁶ lymphoma cells (EL4, ATCC) intramuscularly in hind limbs. Tumors were utilized at a volume of 500-600 mm³. Harvested tumor tissues were fixed overnight in 4% paraformaldehyde (PFA) and then equilibrated overnight in 18% sucrose. Fixed tissues were embedded in optimal cutting temperature (OCT) compound (Sakura Finetek USA, Torrance, Calif.), stored at −80° C. and later cryosectioned at 5 microns per section. Ten tumors of each tumor type (lung, pancreas, melanoma, lymphoma) and at least 5 sections of every tumor were analyzed, totaling 200 slides. The primary antibodies used consisted of 1:500 rat anti-murine CD45 (RDI, Flanders, N.J.), 1:200 rat anti-murine CD31/PECAM-1 (BD Pharmingen, San Diego Calif.), 1:500 rabbit anti-vWF (DAKO, Carpinteria Calif.) and 1:500 rabbit anti-GFP (Novus Biologicals, Littleton Colo.). VWF staining required pre-treatment of sections for 5 minutes at 37° C. with Digest-All 2 (Zymed, San Francisco, Calif.). Vector Labs ABC− Alkaline Phosphatase kit was then used following the manufacturer's instructions, employing Vector Red substrate as the chromagen. CD45 was also detected using this method. GFP could be directly visualized in this system. For CD31 staining, sections were heat retrieved in citrate retrieval buffer pH 6.0 for a total of 50 minutes. Dual staining for CD31 and GFP was then performed. Alexa Fluor donkey anti-rabbit 488, and donkey anti-goat 594 (Molecular Probes, Eugene, Oreg.) diluted at 1:200 were the secondary antibodies. Sections were immunostained with primary antibodies overnight at 4° C., followed by incubation with secondary antibodies for 30 minutes at room temperature. Slides were mounted with Vectashield containing DAPI to allow for nuclear visualization.

### Tissue Analysis

Tumor sections were analyzed using a laser scanning spectral confocal microscope (Leica Microsystems, Bannockburn, Ill.) or an Olympus Provis immunofluorescence microscope (Olympus American, Melville, N.Y.). A total of 2500 blood vessels were examined within all tumors (usually 5-10 stained slides per sample).

### Cytokine and Anti-SDF-1 Treatment

To test the effects of mobilization on marrow hemangioblast activity in cancer slight modifications were made to the transplant model. After transplant and peripheral blood analysis at 3 month to confirm hematopoietic chimerism, mice were divided into 4 groups of 8 (total n=32). All mice received injections of 2×10⁵ LLC cells intramuscularly in hind limbs. One group of mice was a control group and received no treatment after cancer inoculation. Another control group received intratumoral PBS injections. The third group received G-CSF 6 mcg (filgrastim, Amgen) in 100 microliters PBS subcutaneously every day and SCF 100 ng (R&D Systems) in 100 microliters PBS intravenously every third day at a site away from the tumor beginning day −3 and then until day +14 after cancer inoculation. The fourth group received polyclonal anti-SDF-1 antibodies 25 mcg (R&D Systems) in 20 microliters PBS intratumorally every day from day 0 to day +14. Tumors were measured every day using calipers. Volume measurements were calculated based on maximum width and length measurements.

### Example 7

### Human Hematopoietic Cells Contribute to Vascular Repair in Xenograft Model of Vasculopathy

A xenotransplant model was used to determine whether human hematopoietic cells could act as functional hemangioblasts in response to an ischemic challenge. To study adult human hematopoiesis, the nonobese diabetic (NOD)/scid strain has been used because it is tolerant of human chimerism (Shultz et al., J Immunol 154, 180-191 (1995)). Cell enrichment and gene marking studies have shown that the repopulating cells, termed scid mouse-repopulating cells (SRCs), are primitive and distinct (Dick et al., Stem Cells 15, 199-207 (1997)). SRCs are the best surrogate for testing HSCs.

Chimeric mice were generated by irradiating recipient NOD/scid mice with 325 cGy followed by intravenous injection of 2×10⁵ human CD34⁺ cells greatly enriched for HSC/hematopoietic progenitor cell (HPC) from umbilical cord blood (UCB) by magnetic bead positive selection using Miltenyi magnetically activated cell sorter (MACS; Miltenyi Biotech, Auburn, Calif.). The CD34⁺ cells were then stained for CD45 to confirm predominant hematopoietic origin. Typical CD34⁺ purity was more than 95%. At 4 to 6 weeks after xenotransplantation, approximately 50% of NOD/scid mice receiving xenotransplants had human hematopoietic reconstitution in peripheral blood and 80% had multilineage human hematopoietic reconstitution in bone marrow. Production of diploid circulating endothelial progenitor cells (EPCs) (Peichev et al., Blood 95, 952-8. (2000)) was confirmed by sorting either murine or human VEGF receptor 2⁺ (VEGFR-2⁺) cells from the peripheral blood and staining for DNA content with propidium iodide. Both murine-only control animals and xenograft recipients who underwent the neovascularization model had significant levels of circulating diploid EPCs (3%-5% of total peripheral blood mononuclear cells [PBMNCs] versus >0.5% in uninjured control animals). These data strongly suggested that cell fusion does not play a major role in the endothelial cell engraftment detected.

Representative flat mounts of ischemic eyes from 2 animals showed areas with newly regenerated blood vessels that have endothelial cells derived from donor human HSC/HPC. The non-injured control eyes from the same animals have no staining for human CD31, indicating no human contribution to endothelial cells and demonstrating the specificity of the staining protocol. Additionally, immunohistochemistry was performed on sections from fixed eyes to identify human endothelial cell contribution to the newly formed blood vessels within the retina and vitreous of the ischemic eyes. Staining for human-specific CD31 expression was used to detect endothelial cells, whereas staining for human LAMP-1 was used to detect all cells of human origin. Human cells lining blood vessels and expressing endothelial proteins were clearly detected in the ischemic retinas of animals that received xenotransplants. Human endothelial cells were still detectable up to 5 months after xenotransplantation. Overall level of human contribution to neovascularization was in the 1% to 5% range. Control eyes from the same animals demonstrated very low background staining and a complete lack of human cell contribution. The level of human endothelial engraftment in the ischemic eyes roughly correlated with degree of bone marrow chimerism. Without human hematopoietic engraftment human endothelial contribution or circulating EPCs were never observed, strongly suggesting that human HSCs make endothelial progenitor cells. These findings mimic the results of the original murine model.

### Example 8

### Blocking SDF-1 Inhibits Hemangioblast Activity

Within the retina VEGF expression is increased in response to ischemia to promote vascular repair. The primary cytokine involved in homing of HSC to the bone marrow is stromal derived factor-1 (SDF-1) (Wright et al., J Exp Med 195, 1145-54. (2002)). It is likely that SDF-1 acts as a mechanistic regulator of factors required for HSC/EPC recruitment to sites of ischemia. Due to the relative lack of proteases in the vitreous of the eye, SDF-1 leaking into the vitreous was hypothesized to create an artificially high SDF-1 concentration gradient, drawing in HSC/EPC (Sjostrand et al., Acta Ophthalmol (Copenh) 70, 814-9 (1992)).

Blocking SDF-1 should reduce retinal neovascularization from HSC-derived EPC by blocking their recruitment to the site of ischemic injury. To test this hypothesis a murine model was used. To abrogate SDF-1 activity, a cohort of 10 long-term engrafted animals were injected with a SDF-1 specific blocking antibody in PBS (R&D Systems) into the vitreous at the time of laser injury. Weekly booster injections of SDF-1 blocking antibody were given intravitreally during the ischemic repair phase. Two cohorts of 10 animals all with equivalent hematopoietic engraftment received either no intravitreal injections or weekly intravitreal mock injections. Strikingly, the cohort treated with SDF-1 blocking antibody had almost no HSC-derived blood vessels produced in response to VEGF and injury. Cross-sectional histological analysis of treated versus non-treated control eyes were performed to better assess total neovascularization. None of the anti-SDF-1 treated eyes exhibited retinal neovascularization and all retained normal architecture. These results clearly demonstrate that treating the eye with SDF-1 blocking antibody prevents retinal neovascularization.

To determine whether the murine model reflected changes seen in human diabetic retinopathy, human EPC were isolated by FACS (CD34⁺, VEGFR-2⁺ peripheral blood mononuclear cells), placed into trans-well chambers and assayed for migration in response to SDF-1. Human EPC migrate towards physiological concentrations of SDF-1. Human retinal vascular endothelial cells were also cultured. These cells were found to upregulate the expression of VCAM-1 (an important EPC target) in response to SDF-1. Collectively these data demonstrate that SDF-1 plays a critical role in recruiting EPC to the site of ischemic injury. SDF-1 alone can substitute for exogenous VEGF in an adult murine model system. The data from the murine model strongly correlated with and is supported by human patient data. SDF-1, therefore, presents an attractive therapeutic target for blocking and/or promoting angiogenesis. These experiments also highlight the efficacy of our murine model for testing factors or conditions that may affect angiogenesis.

### Example 9

### Leukemia Hemangioblast Activity

To test the ability of leukemia cells to produce endothelial cells human CML blast cells were incubated (K562, ATCC) in EGM-2 media supplemented with 10% fetal bovine serum. After 10 weeks of culture, these human leukemia cell lines developed endothelial colonies that resemble those found during the culture of normal human umbilical cord blood cells. FACS analysis demonstrated a marked increase in expression of endothelial cell surface proteins (CD31, CD141, CD146, and CD34). Furthermore, immunohistochemistry also demonstrated striking increases in expression of the endothelial surface protein, PECAM-1 (CD31) and uptake of LDL (a function of endothelial cells and monocytes).

To test the in vitro hemangioblast potential of leukemia, the K562 leukemia cell line was used, as well as bone marrow samples from two acute myeloid leukemia (AML) patients. Mononuclear cells were collected over a ficoll gradient and then suspended in Matrigel at a dilution of 1×10⁷ cells per mL. Following this resuspension, NOD/scid mice were sublethally irradiated (326 cGy) and then received a subcutaneous injection of the leukemia cells in Matrigel suspension (1×10⁷ cells/mouse). After 4 weeks of growth, the animals (n=3) were sacrificed and Matrigel plugs removed and flash frozen. Sections of the Matrigel were then stained for human specific antigens (LAMP-1). Staining demonstrated blood vessels of human leukemia origin. These blood vessels were predominately located in the periphery of the Matrigel plug.

### Example 10

### Blocking SDF-1 Inhibits Marrow-Derived Microvessel Formation in Cancers

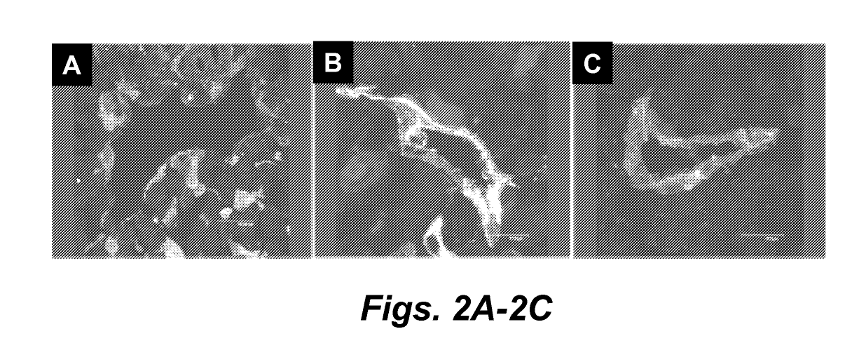

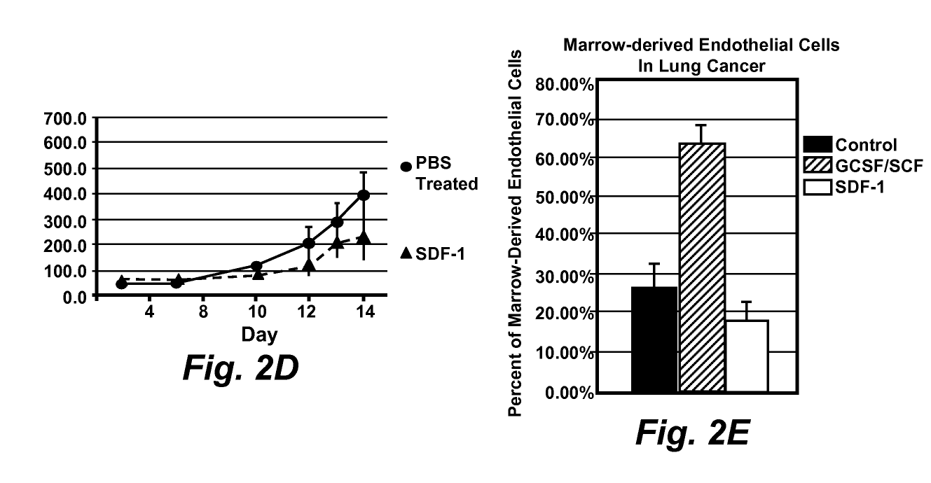

Blood vessel development is needed for cancer growth and metastasis. Based on findings that the hematopoietic stem cell provides functional hemangioblast activity in repairing the ischemic retina, a search for the primary source of tumor vasculature was undertaken. Adult mice were durably engrafted with hematopoietic stem cells from transgenic mice expressing green fluorescent protein. Lung cancers injected in these transplanted mice demonstrated donor marrow-derived blood vessels within the tumor vasculature (FIG. 2). This identified marrow-derived cancer vasculogenesis in settings of melanoma and lymphoma (FIG. 2). To determine whether the tumor neovasculogenesis is from a clonal, self-renewing hematopoietic stem cell lung cancers grown in recipients of single cell and serially transplanted hematopoietic stem cells show clonal, donor-derived blood vessels in 5% of tumor vasculature, matching hematopoietic engraftment. In summary, these results indicated that the self-renewing, clonal adult hematopoietic stem cell exhibits pathologic hemangioblast activity, capable of producing both blood and blood vessels, within tumors (FIGS. 2A-2E). Given that the HSC is involved in hemangioblast activity within cancers, factors affecting leukocyte trafficking are likely to affect hemangioblast activity within cancers. To test this hypothesis, 25 mcg of anti-SDF-1 was injected intratumorally every day for 2 weeks during lung cancer (LLC, ATCC) growth in BL/6 that were previously transplanted with GFP⁺ congenic marrow. The mice that received anti-SDF-1 therapy had markedly reduced microvessel density and lower percentages of marrow-derived blood vessels (FIG. 2A-2E).

### Example 11

### Retinal Ischemic Injury Increased SDF-1α Protein Expression in the Eye

Adult HSC can function as hemangioblasts by producing multilineage long-term engraftment and the generation of newly formed vessels in the retina. Such vessel formation is often associated with proliferative diabetic retinopathy in humans. The formation of BM-derived neovascularization is modulated by and requires SDF-1α, a major chemokine involved in the trafficking of BM-derived cells (Butler et al., J Clin Invest 115:86-93). A murine model of proliferative retinopathy with vascular endothelial growth factor (VEGF) overexpression was used to characterize the role that SDF-1α plays in promoting the recruitment and incorporation of BM-derived EPC to sites of neovascularization.

The murine model used was infected with a recombinant adeno-associated virus type 2 that overexpresses the murine 188 isoform of VEGF-A (rAAV2 VEGF-A 188). Laser-induced ischemic injury was used to promote rampant proliferative neovascularization in adult mice (Grant et al., Nat Med 8:607-612.). By using bone marrow cells isolated from transgenic mice that ubiquitously express DsRed fluorescent protein, the ability of transplanted cells to participate in new blood vessel formation was assessed. A series of variations on this initial model was used to assess various aspects of the neovascularization process.

To determine if SDF-1α is upregulated due to the damage at the sites where preretinal neovascularization is known to occur, and to address the effects of laser-induced ischemic injury over time in the retinal neovascularization model, eyes were harvested at different time points following laser-induced ischemic injury (Day 0—prelaser, 1 Hr, 12 Hrs, Day 1, Day 3, Day 7 and Day 28). Whole eyes were harvested, fixed and sectioned. Immunofluorescene (IF) was used to detect both SDF-1α and HIF-1α protein expression. SDF-1α is known to be constitutively expressed by the retinal pigmented epithelium and secreted into the outer segment layer of the photoreceptors. This basal expression served as an internal positive control for SDF-1α staining for every time point assayed (Photoreceptor Layer (PRL)). The unmanipulated control eyes and the Day 0 eyes appeared to have similar SDF-1α expression patterns as seen by the red fluorescent staining of the photoreceptor layer outer segments. SDF-1α was not detectable in other layers of the retina in the non-laser-damaged eyes. An increase in the level of SDF-1α protein was expressed in the ganglion cell layer (GCL) immediately following ischemic injury. The GCL contains the superficial vascular network of the retina and is the site where VEGF-A is overexpressed by the AAV2 vector. The GCL is also the initiating site for the proliferative neovascularization observed in the mouse model. SDF-1α expression peaked at 1 hour and was maintained until 12 hours post-laser injury. By Day 1 post-laser injury, SDF-1α protein expression had returned to background levels and remained at this level throughout the remaining course of the experiment. Since IF is not an accurate method for quantification, the expression levels of SDF-1α in the vitreal space of the eyes was measured by ELISA. The ELISA showed a direct correlation with the IF for SDF-1α expression levels.

IHC was also performed to detect the transcription factor hypoxia inducible factor-1 alpha (HIF-1α). Recent evidence has shown that SDF-1α gene expression was regulated by HIF-1α in endothelial cells, resulting in selective in in vivo expression of SDF-1α, which is directly proportional to reduced oxygen tension (Ceradini et al, Nat Med 10:858-864). The C57/BL6 control animals did not show any expression of HIF-1. On Day 0 an increase of HIF-1α protein in the GCL was observed. One month prior to Day 0, with respect to laser injury, the test eye was injected with a rAAV2 VEGF-A 188. As described previously (Grant et al., Nat Med 8:607-612), Day 0 with respect to laser injury was chosen to coincide with peak expression of VEGF-A in order to induce maximal proliferative neovascularization. The HIF-1α expression seen immediately prior to laser injury suggested that overexpression of VEGF-A was sufficient to induce HIF-1α expression. The HIF-1α protein was predominantly localized in the cytoplasm, suggesting that HIF-1α was not activated. HIF-1α activation resulted in its translocation from the cytoplasm to the nucleus and was in close proximity for binding to target promoter regions, such as the VEGF-A and SDF-1α promoters in ischemic tissues. By 1 hour post-injury, the same relative and expression pattern of HIF-1α was observed as compared to Day 0. At best a few cells may have shown some evidence of HIF-1α translocated to the nucleus, but the effects observed were minimal. Overall, HIF-1α protein expression was maintained in the GCL for every additional time point analyzed. Without wishing to be tied to theory, these data indicate that the exogenous expression of VEGF A in the murine model of retinal neovascularization induced constant expression of HIF-1α in the retinal GCL without inducing SDF-1α expression until after the laser injury occurred. Laser-induced ischemic injury likely activated HIF-1α translocation to the nucleus, where it plays a role in upregulating SDF-1α expression. Translocation of HIF-1α was extremely modest and HIF-1α expression remained high at 1 day post-laser when SDF-1α is no longer detectable in the ganglion cell layer. These data suggest that HIF-1α may be permissive for but not directly regulate SDF-1α expression/maintenance.

### Example 12

### SDF-1α Localized to the Bone Marrow Vascular Niche Following Retinal Ischemic Injury

The original source of EPC that participate in the repair/production of blood vessels in ischemic tissues is believed to be the bone marrow. To participate in this repair, BM-derived cells must migrate from the bone marrow to the peripheral blood. This process is accomplished by transendothelial migration of BM-derived cells through the sinusoidal endothelium that are present throughout the vascular niche of the bone marrow compartment. To determine whether SDF-1α protein levels were increased in the bone marrow compartment following retinal ischemic injury, the murine retinal injury model was used, and bones harvested at the same time points as the eyes were analyzed. Immunofluorescence (IF) was used to detect SDF-1α protein expression. The overexpression of VEGF-A in the eye by rAAV VEGF-A expression vector had no effect on the expression pattern of SDF-1α in the marrow as compared to the control bones. At 12 hours, the bone marrow vascular niche expressed SDF-1α. By Day 1, nearly all areas of the vascular niche expressed SDF-1α. SDF-1α expression returned to control levels by Day 3. Similar expression patterns were seen at all time points, except for the 12 hour and Day 1 time points. In order to quantify the expression of SDF-1α ELISA was performed on bone marrow extracts. Once again, a correlation was observed between the IF expression pattern and quantified protein levels of SDF-1α.

### Example 13

### CD133⁺CXCR4⁺ Cells Contained HSC-Derived Proangiogenic Progenitors

### “Effector” EPC

The cell surface marker CD133 was originally described on human cells as a marker of early stem/progenitor cells. The human CD133⁺ cell population has been shown to contain HSC (as measured by SCID repopulating cells) (Wognum et al., Arch Med Res 34:461-475) and EPC. CD133 is expressed only on very immature endothelial progenitor cells and its expression is lost as the endothelial cells mature. The murine homolog of CD133 has recently been identified. Murine CD133 has approximately 60% homology to human CD133. To determine if murine CD133 is expressed on long-term repopulating HSC in the mouse, lethally-irradiated (950 rads) female C57/B16 mice (n=10) were transplanted with 1×10⁶ male CD133⁺DsRed⁺ cells (n=5) or 2000 male S⁺K⁺L⁻DsRed⁺ cells (n=5), which served as a control. All mice were co-transplanted with a radioprotective dose consisting of 1×10⁵ syngeneic whole bone marrow cells to ensure long term survival. All mice transplanted with CD133⁺DsRed⁺ cells showed no hematopoietic engraftment at one and three months post-transplant, while all mice in the control cohort exhibited long-term hematopoietic engraftment. Therefore, CD133⁺ bone marrow cells did not provide long-term HSC activity in the mouse.

The murine adult HSC was also able to function as a hemangioblast in vivo, contributing both to blood reconstitution and to blood vessel repair in response to ischemic injury by producing circulating EPC. SDF-1α is known to be required for recruitment of EPC (Butler J Clin Invest 115:86-93), therefore CD133 bone marrow cells were examined for the expression of CXCR4, the receptor for SDF-1α, by flow cytometry along with a variety of progenitor/lineage markers. This study addressed whether the CD133 population contained the “effector” EPC population that directly participate in neovascularization. Since the murine EPC is functionally defined as the circulating BM-derived cells that participate in new blood vessel formation/vessel repair, the peripheral blood was examined for CD133⁺CXCR4⁺ cells. CD133⁺CXCR4⁺ cells were found to constitute approximately 4% of the mononuclear cells compared to approximately 7% of the bone marrow. Regardless of origin, the majority of CD133⁺ cells expressed CXCR4 and migrated toward SDF-1α in a dose-dependent manner, suggesting that SDF-1α may act as a recruiting chemokine for a putative CD133⁺ EPC in vivo.

CD133⁺CXCR4⁺ cells expressed hematopoietic progenitor cell surface markers, such as CD45, CD117 (c-kit), Sca-1, VLA-4, CD11b, CD44 and CD135 (flt-3). They expressed lower levels of VEGFR2 and CD31, usually associated with EC, but also expressed the primitive hematopoietic marker CD150. The mature endothelial cell markers VE cadherin and Tie 2 were not markedly expressed. Therefore, the CD133⁺CXCR4⁺ cells uniformly expressed all of the suggested markers associated with EPC in the murine system. These data suggested that BM-derived CD133⁺CXCR4⁺ cells that are myleomonocytic and endothelial-like may have proangiogenic potential.

### Example 14

### SDF-1α-Mediated Mobilization of BM-Derived CD133⁺CXCR4⁺ Cells Following Retinal Ischemic Injury

To further establish that BM-derived CD133⁺CXCR4⁺ cells constitute a functional “effector” EPC population, their levels were assayed in the peripheral blood during the time course assayed in the murine neovascularization model. Using flow cytometry, an initial increase of CD133⁺CXCR4⁺ cells on Day 0 versus wild-type controls was observed. This increase was likely caused by an increase in VEGF-A in the plasma following the intravitreal injection of rAAV VEGF-A 188. Following laser-induced ischemic injury, there was a sustained increase of CD133⁺CXCR4⁺ cells from 12 hours to 3 Days.

To determine if the increase in circulating BM-derived CD133⁺CXCR4⁺ cells was mediated by SDF-1α, plasma was collected at the same time points described above. ELISA was used to assay the plasma. A correlation was observed between the percentage of CD133⁺CXCR4 cells circulating in the peripheral blood and the level of SDF-1α protein. A comparison of the SDF-1α ELISA results of the bone marrow and plasma samples showed SDF-1α accumulation that likely promotes the mobilization of CD133⁺CXCR4 cells from the bone marrow to the peripheral blood.

To further substantiate that the SDF-1α/CXCR4 axis is responsible for the mobilization of CD133⁺CXCR4⁺ cells to the peripheral blood, the hematopoietic cytokine soluble Kit ligand (sKitL), also known as stem cell factor (SCF) was used. sKitL has been shown to be necessary for mobilization of hematopoietic cells and can exert a proangiogenic effect on human umbilical vein endothelial cells. sKitL has also been shown to increase plasma levels of SDF-1α (Grant et al., Nat. Med. 2002; 8:607-612). If sKitL does in fact increase plasma SDF-1α, it is likely that an intravenous (i.v.) injection of sKitL would mobilize CD133⁺CXCR4⁺ cells from the bone marrow. Therefore, blocking SDF-1α or CXCR4-mediated signaling was expected to disrupt the ability of sKitL to mobilize these cells. sKitL was found to have a profound effect on mobilization, resulting in approximately a 4-fold increase of CD133⁺CXCR4⁺ cells in the peripheral blood at Day 3 post-injection as compared to the IgG isotype or PBS controls. Using neutralizing antibodies to block CXCR4 (clone 2B11) (41) or SDF-1α (MAB 310) (Butler et al., 2005 J Clin Invest 115:86-93). signaling resulted in the inhibition of sKitL-mediated mobilization of CD133⁺CXCR4⁺ cells (FIG. 12C). These data indicated that elevation of SDF-1α in the plasma supports the mobilization of CD133⁺CXCR4⁺ cells, in part by activating the signaling cascade of the SDF-1α/CXCR4 axis.

### Example 15

### BM-Derived CD133⁺CXCR4⁺ Participated in Blood Vessel Formation In Vivo

To determine if BM-derived CD133⁺CXCR4⁺ cells could actively participate in neovascularization, the standard murine model of proliferative diabetic retinopathy was modified. The model was modified by adoptively transferring fluorescently tagged donor cells one day after laser injury in order to test for short-term “effector” cell activity. Briefly, C57BL/6 mice were injected with rAAV2 VEGF-A 188 in the right eye (n=30). After four weeks, when VEGF-A expression peaked, laser photocoagulation was performed on the right eyes in order to promote neovascularization. The day following ischemic injury, CD133⁺CXCR4⁺DsRed⁺ cells were isolated from the bone marrow of donor mice and 1×10⁶ cells were infused i.v. via the retro-orbital sinus into the prepared cohorts (n=6). Right and left eyes were enucleated and retinas were flat mounted four weeks post laser injury. All left eyes showed no contribution from the CD133⁺CXCR4⁺DsRed⁺ donor cells. However, right eyes that received both VEGF and laser treatment (FIGS. 13A,B, BM-derived CD133.CXCR4.DsRed+) showed extensive contribution from the CD133⁺CXCR4⁺DsRed⁺ donor cells to sites of neovascularization. CD133⁺CXCR4⁺DsRed⁺ directly participated in vessel formation by forming functional endothelium and large, nonfunctional endothelial-like tubes, which may act as a scaffold for the newly forming vasculature. These test retinas were indistinguishable from the standard retinal neovascularization model control, containing long-term engrafted S⁺K⁺L⁻DsRed⁺ HSC (>4 months) contributing to neovascularization as expected (Grant et al., Nat. Med. 2002; 8:607-612; Butler et al., 2005 J Clin Invest 115:86-93; Guthrie et al., 2005 Blood 105: 1916-1922). If CD133⁺CXCR4⁺DsRed⁺ cells are effector EPC, they should participate in perivascular and/or lumenal incorporation. Therefore, retinal flat mounts were analyzed for the presence of such cells at high magnification. The CD133⁺CXCR4⁺DsRed⁺ were found to directly participate in vessel formation by incorporating into the vessel (as shown by colocalization of FITC Dextran and DsRed). These cells localized to the periendothelial region of the lumen in the newly formed vessels. These data indicated that BM-derived CD133⁺CXCR4⁺ are bona fide “effector” EPC that are recruited to the sites neovascularization to directly participate in vessel formation.

### Example 16

### Anti-SDF-1α Antibody Blocked Recruitment of CD133⁺CXCR4⁺DsRed⁺ Cells to Sites of Preretinal Neovascularization

These data indicate that SDF-1α plays a major role in the recruitment of effector EPC cells to sites of preretinal neovascularization. In order to determine if SDF-1α is necessary for the function of this subpopulation, CD133⁺CXCR4⁺DsRed⁺ cells were isolated and adoptively transferred (1×10⁶ per recipient animal) one day following retinal ischemic injury in the modified model. Concurrently with the cell infusion, neutralizing antibodies to either SDF-1α (clone MAB301) or CXCR4 (clone 2B11) were injected into the vitreous of separate cohorts of mice (n=6). In the eyes that received the anti-SDF-1a antibody, there was maximal blockage of recruitment and incorporation at the sites of vascular injury, whereas in the eyes that received the anti-CXCR4 antibody, there was recruitment to the sites of injury, but no incorporation of CD133⁺CXCR4⁺DsRed⁺ cells into new vessels. IgG control eyes showed a minimal block in contribution from the CD133⁺/CXCR4⁺/DsRed⁺ donor cells to sites of neovascularization. In order to quantify the effectiveness of the blocking antibody treatments photomicrograph montages on each treated retina (n=6 per cohort) were assembled using the 20× objective on a Leica TCS spectral confocal microscope. Volocity Image Quantification software (Perkin Elmer Inc.) was used to calculate the relative contribution of dsRED to FITC fluorescence area which is reported as percent DsRed positive cells in the retinal vasculature. These data indicated that the SDF-1α/CXCR4 axis was required for successful contribution of “effector” EPC at sites of ischemic injury.

### Example 17

### CD133⁺CXCR4⁺ “effector” EPC Differentiated into Smooth Muscle-Like Cells

The periendothelial location of CD133⁺CXCR4⁺ effector EPC in larger vessels suggested that an enriched “effector” EPC may also be capable of differentiating into smooth-muscle like cells. After adoptively transferring CD133⁺CXCR4⁺DsRed⁺ (1×10⁶ per recipient animal, n=6) one day following retinal ischemic injury and allowing for neovascularization, retinas were stained for smooth muscle actin (SMA, in blue). A proportion of the functional vasculature that expressed smooth muscle actin were also positive for DsRed “effector” EPC. Quantification, via photomicrography and image analysis using Volocity software, of the treated retinas clearly shows that a significant proportion of CD133⁺CXCR4⁺DsRed⁺ that contribute to the injured vasculature also express SMA. Without wishing to be bound by theory, these data indicate that CD133⁺CXCR4⁺ “effector” EPC are recruited to the sites of retinal injury where they not only directly participate in new vessel formation, but also differentiate into supporting cells that express SMA.

### Example 18

### Anti-SDF-1α Treatment for Proliferative Retinopathy is Efficacious in Non-Human Primates

Previous studies have described both the use of laser induced ischemic injury or AAV-VEGF to induce proliferative retinopathy in Rhesus macaques (Lebherz 2005 Diabetes 54:1141-1149; Tolentino 2002 Am J Ophthalmol 133:373-385). To test the efficacy of the anti-SDF-1α therapy in a non-human primate model of proliferative retinopathy, these two models were combined in Rhesus as described in the rodent model of proliferative retinopathy. The following treatment groups were compared: controls, which received no manipulation, VEGF plus laser, VEGF plus laser with isotype antibody administration, or VEGF plus laser with anti-SDF-1α antibody administration. As in the rodent model, eyes were harvested one month after laser injury for analysis. The macaques were not transplanted with tagged cells due to limitations in availability and the desire to use as few subjects as possible. Previous studies in Rhesus indicated that the majority of neovascularization would occur within the retina itself with a small percentage of preretinal vessel formation also occurring. Therefore, untreated and treated eyes were harvested, fixed in formalin, then sectioned and stained with Hematoxilin and Eosin (H+E) for retinal structure and with antibody to the endothelial marker CD31 to definitively identify blood vessels.

When compared to control retinas, H+E staining of retinas that received AAV2-VEGF plus laser photocoagulation clearly showed growth of large vessels at the sites of laser injury and disruption of retinal architecture. A large vessel was qualitatively defined as one that plainly contained red blood cells within its lumen. When anti-SDF-1 antibody was provided in addition to AAV2-VEGF and laser, laser burns were still clearly visible, however new vessels failed to form at the burn sites. This observation held true even at sites of severe laser burns, where the retinal architecture was significantly disrupted. Quantification of the number of large vessels per retinal section demonstrated a significant increase in retinal vessels in the AAV2-VEGF plus laser-treated eyes (mean 21+/−4 vessels per section) compared to control eyes (mean 3+/−3.5 vessels per section). Eyes treated with anti-SDF-1 in addition to AAV2-VEGF and laser were found to be no different than untreated control eyes in the number of large vessels present (mean 4+/−2.5 vessels per section).

In order to confirm neovascularization, retinal sections were stained for the endothelial marker CD31. CD31 staining clearly outlined large vessels within the retina and pre-retinal (vitreal) vessels in AAV2-VEGF-plus-laser-treated eyes. In contrast, vessels in retinas of eyes that also received anti-SDF-1α antibody were rare, quite small and/or represented the remains of vessels that had been subject to photocoagulation, even when severe laser burns were present. No pre-retinal vessels were found in treated eyes that also received anti-SDF-1α antibody. Yerkes performed a full autopsy and toxicology report on each animal and no systemic or adverse effects of intravitreal anti-SDF-1α treatment were apparent at any time during the study.

### Example 19

### Anti-SDF-1α Treatment was Efficacious in Models of Intimal Hyperplasia and Tumor Neovascularization

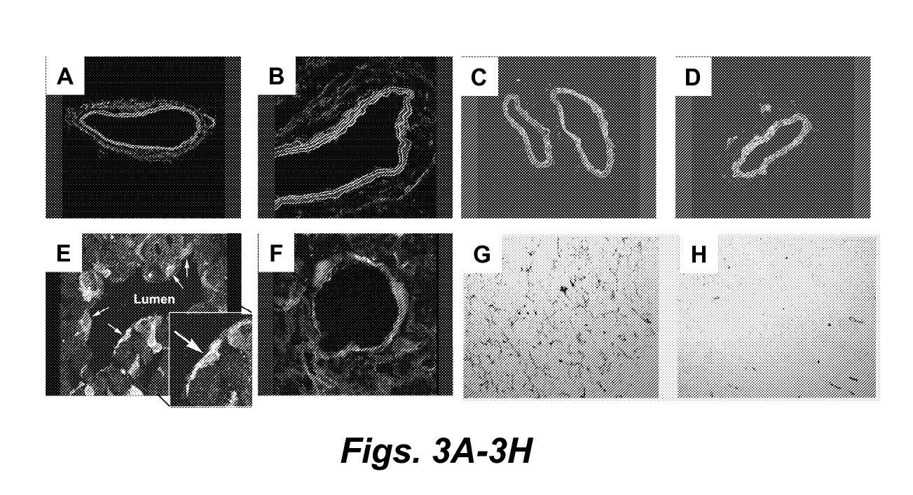

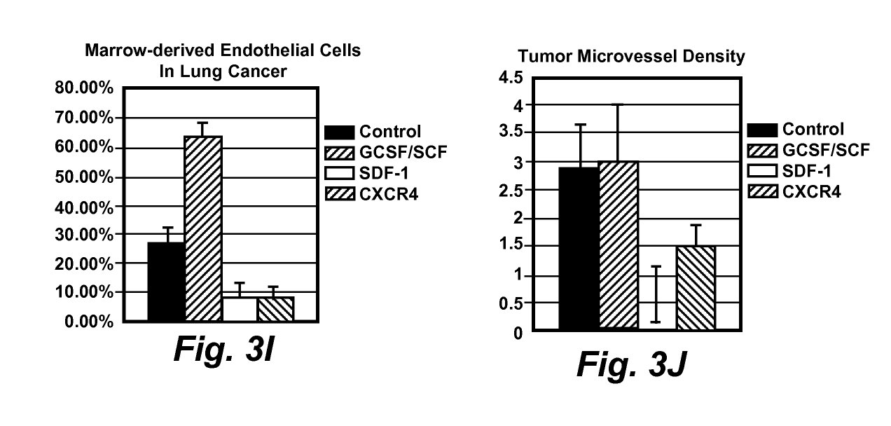

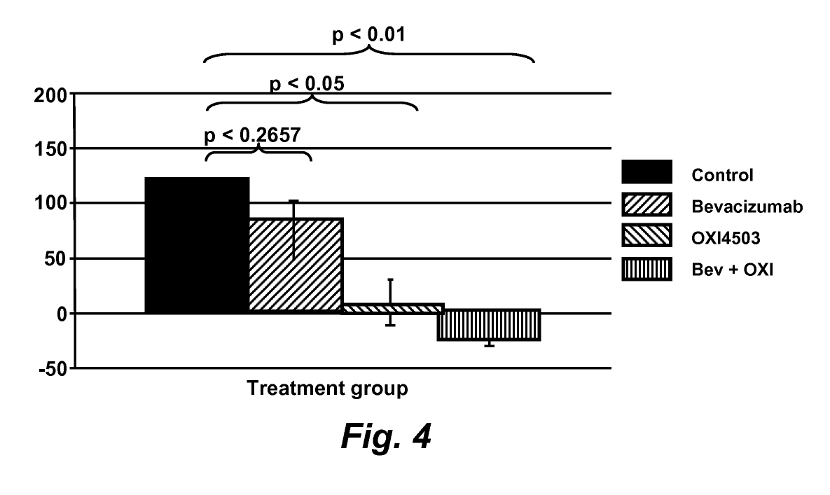

Bone marrow derived progenitors play a major role in intimal hyperplasia in a mouse model (Diao et al., Am J Pathol 172:839-848). This model was used to test the effectiveness of anti-SDF-1α therapy in preventing intimal hyperplasia. Cohorts of mice were transplanted with DsRed⁺ enriched HSC and confirmed for long-term engraftment after three months. One cohort underwent the intimal hyperplasia model as before and received injections of isotype control antibody at the site of injury (25 μg every other day for 8 days). As shown previously, DsRed⁺ BM-derived cells clearly played a major role in the resulting hyperplasia (FIGS. 3A,B). A second cohort underwent the intimal hyperplasia model, but also received anti-SDF-1α antibody, which was formulated in a timed release disc implanted adjacent to the graft (5 ug antibody released per day for 2 weeks). The test vessels in animals that received anti-SDF-1α antibody showed little if any hyperplasia and lacked significant DsRed⁺ BM-derived contribution to the vessel (FIGS. 3C,D). Therefore, anti-SDF-1α antibody effectively prevented marrow-derived contributions to intimal hyperplasia.

### Example 20

### Anti-SDF-1α Treatment Inhibited Tumor Angiogenesis

A model of tumor neovascularization was used to test the efficacy of anti-SDF-1α antibody at slowing or preventing tumor growth. To analyze the clonal HSC-derived nature of the tumor hemangioblast activity, single GFP⁺ HSC were engrafted long-term into primary recipient animals as described previously (Grant et al, Nat Med 8:607-612). Gfp⁺, Sca-1⁺, c-kit⁺, Lin⁻ HSC were isolated from the primary recipients and transplanted into secondary cohorts as serial transplants. The serial transplant recipients were monitored for long-term engraftment and then inoculated subcutaneously in the hind limb with 2×10⁵ C57B6 derived Lewis lung carcinoma cells, which were allowed to form tumor masses for two weeks. The tumors were then harvested, fixed, sectioned and stained for CD31 to demarcate vascular endothelium (FIG. 3E, native Gfp and RED CD31 IF). 25% of the identifiable vessels within each tumor section contained donor-HSC derived CD31⁺ cells with classic endothelial morphology upon confocal microscopy (FIG. 3E arrows, I). The expanded single HSC serial transplant experiments were confirmed with additional cohorts of SKL-enriched, DsRed⁺ HSC recipients with LLC tumors (FIG. 3F, native DsRed and GREEN CD31 IF).

Given the bipotentiality of HSC in producing both blood and blood vessels within cancer, it is likely that factors involved in leukocyte trafficking regulate contribution to cancer blood vessels. To test this hypothesis, a slight modification was made to the tumor model. After GFP transplant and lung cancer inoculation, cytokines involved in marrow cell mobilization (i.e., granulocyte colony stimulating factor (G-CSF) and stem cell factor (SCF) were administered. Over the course of fourteen days, one cohort of mice (n=8) received daily subcutaneous G-CSF and every third day SCF was administered intravenously. At the end of 14 days, all mice demonstrated elevated white blood counts. One control cohort of mice received no injections (n=4). Another control cohort received intratumoral PBS injections (n=4). Under these conditions, lung cancer growth in all animals was measured daily. Over 14 days, the tumors in the G-CSF and SCF treated group grew at a faster rate and to a larger average size of 600+/−67 mm³ than tumors in the control/PBS injected mice, where tumor size averaged 325+/−28 mm³. In cytokine treated animals, microvessel density was not different compared to control mice (FIG. 3G for example, quantified in J). Interestingly, marrow-derived cells in the wall of tumor blood vessels was markedly elevated in the cytokine treated mice compared to control mice (63% vs. 26%) (FIG. 3I).

In view of data reported herein indicating that the SDF-1/CXCR4 axis is important for marrow cell homing and migration, blocking this axis was expected to block marrow-derived blood vessels in cancer. To test this hypothesis, GFP marrow was transplanted into wild-type C57BL/6 mice and these mice were inoculated with lung cancer (n=8). These mice were treated with anti-SDF-1α or anti-CXCR-4 antibodies injected intratumorally every day for 14 days. Over the ensuing 14 days, tumors in the anti-SDF-1α treated group grew at a much slower rate and to a much smaller size, average 125+/−16 mm³. Tumors in the anti-CXCR-4 treated group grew to an intermediate average size of 230+/−18 mm³. Microvessel density was markedly decreased in the anti-SDF-1α cohort and significantly decreased in the anti-CXCR-4 treated cohort compared to the control cohorts (FIG. 3G for example of anti-SDF-1α treated tumor, quantified in J). Moreover, marrow contribution to tumor neovessels was significantly decreased in the anti-SDF-1α/CXCR-4 treated tumors compared to controls (FIG. 3I).

The anti-neoplastic effect of anti-SDF-1α/CXCR-4 antibodies could be due to a direct cytotoxic effect of the antibodies on growing cancer cells. To address this possibility, in vitro cultures of LLC lung cancer cells were established in the presence of escalating concentrations of anti-SDF-1α antibodies. Concentrations equal to or above the levels used in the in vivo animal experiments were used. In vitro cell growth was similar between all cultures, and at all antibody concentrations tested. These results indicate that the anti-SDF-1α antibody was not cytotoxic. Thus, in tumors as in retinopathy, anti-SDF-1α treatment exhibited significant anti-angiogenic effects. The anti-angiogenic effects retarded tumor growth indicating that anti-SDF-1α treatment would be efficacious for the treatment of cancer.

Results described in Examples 11-20 were carried out using the following methods and materials.

Mice: C57BL/6 mice were purchased from Charles River Laboratories. C57BL/6 mice that ubiquitously express DsRed.MST under the control of the chicken B-actin promoter and CMV enhancer were obtained from The Jackson Laboratory (Bar Harbor, Me.). The Gfp⁺ mice are from STOCK Tg(GFPU)5Nagy/J (The Jackson Laboratory) mice. Primates: Rhesus macaques were purchased and housed at the Yerkes National Primate Center (Atlanta, Ga.).

C57BL/6.DsRed radiation chimeric mice were generated by irradiating recipient C57BL/6 mice with 950 rads followed by retro-orbital injection of 2000 Sca-1⁺ckit⁺Lineage⁻ enriched HSC from DsRed⁺ or Gfp⁺ mice and a radioprotective dose of 2×10⁵ Sca-1 depleted bone marrow. HSC were enriched from adult bone marrow as follows: marrow was flushed from long bones, made into a single-cell suspension and plated onto treated plastic dishes in IMDM+20% FBS for 4 hours. Non-adherent cells were collected and 3 rounds of lineage antibody depletion (B220, CD3, CD4, CD8, CD11b, Gr-1 and TER 119) was performed with the Milteyni MACS (Auburn, Calif.) system until a small aliquot stained with PE-conjugated lineage-antibody mixture showed 95% lineage-negative by FACS. The Lin⁻ cells were then positively selected for Sca-1 and c-kit and were sorted using a FACSVantage SE. Mice were checked for multilineage engraftment using flow cytometry (FACSCalibur, BD Biosciences, San Jose, Calif.) 3 months post irradiation using monoclonal antibodies against CD11b, B220, and CD3e conjugated to PE (BD PharMingen, San Diego, Calif.). Single HSC transplants were performed as described previously (Grant et al., Nat Med 8:607-612, 2002) to establish clonality. Long-term engrafted primary clonal recipients served as marrow donors for cohorts of secondary transplants for tumor inoculation and subsequent analysis.

Mouse circulating mononuclear cells were labeled with the following monoclonal antibodies: PE-conjugated and FITC-conjugated CD133-specific (clone 13A4) and Biotin-conjugated Tie-2 (TEK4) from eBiosciences; purified and FITC-conjugated CD184-specific (2B11/CXCR4), PE-conjugated CD45.1-specific (A20), PE-conjugated CD117-specific (2B8), PE-conjugated Sca-1-specific (D7), PE-conjugated CD135 (A2F10.1), PE-conjugated CD11b-specific (M1/70), PE-conjugated CD31-specific (PECAM-1), PE-conjugated flk-1-specific (VEGF-R2) from BD Pharmingen CD150:ALEXA 647 from Serotec; and PE-conjugated CD146-specific (Ms X Endothelial Cells) from Chemicon International. Secondary antibodies: Streptavidin-PE and APC labeled goat anti-rat from BD Pharmingen.

For mice, the standard retinal neovascularization model was performed as described previously (Grant Nat Med 8:607-612, 2002). Briefly, wild-type C57BL/6 mice or mice with >85% donor derived engraftment (compared to wild type GFP or DsRed peripheral blood mononuclear cells) were injected intra orbitally (i.o.) with adeno-associated virus serotype 2(AAV2)-VEGF-A murine 188 in their right eye. One month following AAV2-VEGF-A 188 administration, mice were anesthetized with avertin (2,2,2-tribromoethanol; 240 mg/kg) and injected with 10% fluorescein to facilitate visualization of retinal blood vessels. An argon green laser system (HGM Corporation, Salt Lake City, Utah) was used for retinal vessel photocoagulation with the aid of a 78-diopter lens. The blue-green argon laser (wavelength 488-514 nm) was applied to selected venous sites next to the optic nerve. Venous occlusion was accomplished using laser parameters of 1-second duration, 50-m spot size and 50-100-mW intensity.

One month following laser ablation, mice were deeply anesthetized intraperitoneally with avertin and perfused via the left ventricle with 3 ml of 4% paraformaldehyde in PBS containing fluorescein isothio-cyanate-(FITC) dextran (10 mg/ml, MW 70000, Sigma). Eyes were enucleated and placed in fresh 4% PFA for 60 minutes at room temperature. After washing in PBS, retinas were removed, mounted flat, counterstained and mounted in Vectashield (Vector Labs) with 4′-6-diamidino-2-phenylindole (DAPI).

For primates, a small-scale study in Rhesus macaques was performed to recapitulate our model and to test the safety and effectiveness of anti-SDF-1 treatment. To induce retinal neovascularization in Rhesus macaques procedures used in the murine model was scaled as follows: Matched cohorts (n=5) were injected with saturating titers (5×10¹¹ PFU) of Adeno-Associated Virus-2 (AAV, Vector Core, University of Florida) expressing VEGF directly into the vitreous using a 26-gauge needle and Hamilton syringe. One month after viral infection, the test eyes underwent laser treatment. An argon green laser system (HGM Corporation, Salt Lake City, Utah) was used for retinal vessel photocoagulation with the aid of a 28-diopter lens. The blue-green argon laser (wavelength 488-514 nm) was applied to various venous sites juxtaposed the optic nerve. The venous occlusions were accomplished with >80 burns of 1-sec duration, 150 micron spot size, and 50-100 mW intensity. Venous occlusion were readily visualized as a loss of downstream circulation resulting in a whitening of the vessel and cessation of circulating fluorescent dye administered pre-treatment into the bloodstream. The venous occlusion targets larger vessels in a semi-circle arc around the retinal disk in order to establish ischemia in approximately one half of the retina.

Peripheral blood from DsRed.MST transgenic mice was isolated and the mononuclear cell fraction was collected with Ficoll Paque (Amersham Biosciences) centrifugation purification. The mononuclear cells were washed in 5× volumes of PBS. The mononuclear layer was then resuspended in 100 microliters of PBS and stained with monoclonal antibodies: rat anti-mouse monoclonal antibodies directed against CD133 (clone 13A4; FITC conjugate) and CD184/CXCR4 (clone 2B11), which was detected with a APC-conjugated goat anti-rat IgG antibody (BD Pharmingen). The cells were sorted using the FACSvantage SE for CD133⁺CXCR4⁺DsRed⁺ cells.

CD133⁺CXCR4⁺DsRed⁺ cells were sorted the day after mice underwent vessel photocoagulation. The mice were anaesthetized and 1×10⁶ CD133⁺/CXCR4⁺/DsRed⁺ cells were infused into the retro-orbital sinus of the mice. DsRed⁺ radiation chimeras received vein grafts which were harvested after two weeks, fixed and sectioned as described previously (Diao et al., Am J Pathol 172:839-848, 2008).

To test the effects of mobilization on marrow hemangioblast activity in cancer, slight modifications were made to the transplant model. After transplant and peripheral blood analysis at 3 months to confirm hematopoietic chimerism, mice were divided into 4 groups of 8 (total n=32). All mice received injections of 2×10⁵ LLC cells intramuscularly in hind limbs. One group of mice was a control group and received no treatment after cancer inoculation. Another control group received intratumoral PBS injections. The third group received G-CSF 6 mcg (filgrastim, Amgen) in 100 microliters PBS subcutaneously every day and SCF 100 ng (R&D Systems) in 100 microliters PBS intravenously every third day at a site away from the tumor beginning day −3 and then until day +14 after cancer inoculation. The fourth group received polyclonal anti-SDF-1α antibodies 25 mcg (R&D Systems) in 20 microliters PBS intratumorally every day from day 0 to day +14. Tumors were measured every day using calipers. Volume measurements were calculated based on maximum width and length measurements.

Animals were sedated and perfused through the left ventricle with 4% paraformaldehyde. Immediately following the perfusion, the long bones in the hind limbs were removed and the eyes were enucleated by sliding a curved forcep behind the eyeball and pulling the globe out. Both bones and eyes were immediately placed in 4% PFA and placed in 4° C. refrigerator overnight. Bones and eyes were transferred to 70% ethanol and placed in 4° C. refrigerator overnight. Bones were decalcified and both bones and eyes were embedded in paraffin. Samples were sectioned using a Microm sectioning apparatus at a thickness of 5 microns and placed on microscope slides. Slides were left to dry overnight. Slides were then pretreated for deparaffinization and retrieval of the antigens of interest (SDF-1α and HIF-1α). For the neural retina, heat retrieval with Citrate Buffer was used for antigen retrieval of HIF-1α and SDF-1α. For antigen retrieval of SDF-1α in the long bones, the slides were placed in a 37° C. water bath overnight with Target Retrieval Solution, High pH (pH 9.9, Dako). Slides were washed twice with Tris/Saline Buffer and blocked with Horse Serum for 20 minutes. Primary antibodies pAb anti-SDF-1α Santa Cruz C-19) and pAb anti-HIF-1α (Novus NB100-449) were used at a 1:40 dilution and incubated at 4° C. overnight. Slides were washed three times with Tris/Saline Buffer. Slides were placed at room temperature and washed 3× for 5 minutes with Tris/Saline buffer. Excess buffer was blotted and slides were stained with fluorescent anti-primary species secondary antibodies (Donkey anti-Goat 594-alexafluor for SDF-1α and Donkey anti-Rabbit 488-alexafluor for HIF-1α). Fluorescent secondary antibodies were diluted 1:200 using Zymed diluent and stained for 60 minutes in the dark. Slides were washed 3× for 3 minutes using Tris/Saline buffer at room temperature. All excess buffer was removed and coverslips were mounted with Vectashield with DAPI.

The Rhesus eyes were fixed in formalin, sectioned and stained with Hematoxilin and Eosin (H+E) for retinal structure and with antibody to the endothelial marker CD31 to definitively identify blood vessels (Human CD31, BD).

For tumor neovascularization 6 micron fixed frozen sections were blocked and stained and washed as above using primary antibodies for murine CD31 (BD) with a FITC conjugated secondary antibody (Vector) or with a cocktail of CD31, vWF and MECA-32 antibodies (BD) followed by a DAB conjugated secondary (Vector).

Retinal flat mounts were blocked for 4 hours with 10% Normal Donkey Serum in PBS with 0.3% Triton X. 1:100 dilution of mouse anti-Human SMA (Dako USA) in 10% Normal Donkey Serum in PBS with 0.3% Triton X was used to stain retinas over night. The retinas went through six 1 hour washes in PBS with 0.3% Triton X. 1:150 dilution of Donkey anti-Mouse CY5 in 10% Normal Donkey Serum in PBS with 0.3% Triton X was used to stain retinas over night. The retinas went through six 1 hour washes in PBS with 0.3% Triton X and cover slipped using Hardmount Vectashield without Dapi (Vector Laboratories).

The tissues that were collected for the detection of SDF-1α by ELISA included bone marrow, plasma and vitreous fluid. Erythrocytes were removed from the whole bone marrow by a Ficoll Paque (Amersham Biosciences) purification. Briefly, the bone marrow/PBS sample was layered on top of two times greater volume of Ficoll. The emulsion was centrifuged and the “buffy” layer containing the nucleated cells at the interface was harvested. The mononuclear layer containing the nucleated cells was washed in 5× volumes of PBS. The nucleated cells were then counted using a hemacytometer. 2.5×10⁵ cells were collected from each animal. Cells were resuspended in 500 μl of a protease cocktail inhibitor (BD Biosciences)/PBS solution. Cells were sonicated using a Sonifier 450 (Branson) for 2 seconds (20% duty cycle at level 4 output control). Samples were immediately placed at −80° C. until time of analysis. Plasma was collected by isolating peripheral blood from the retro-orbital plexus and mixing it with PBS containing 10 mM EDTA as an anticoagulant. Samples were centrifuged at 1,000 r.p.m. at 24-27° C. for 5 min and the plasma was harvested in the form of a supernatant. Samples were immediately placed at −80° C. until time of analysis. Vitreous fluid was collected by anaesthetizing the mice and using a 36-gauge needle and Hamilton syringe. The needle was placed directly into the vitreous and 5 μl of vitreal fluid was removed. The fluid was placed in a 1.5 mL collection tube. Forty five μl of PBS was added to the tube for a final volume of 50 μl. Samples were immediately placed at −80° C. until time of analysis. All samples were analyzed for SDF-1α using ELISA (R&D Systems). ELISA assay for SDF-1α was performed according to the manufacturer's instructions (R&D Systems).

Mice: Immediately following laser photocoagulation, as described above, mice underwent intravitreal injections into the right eye or injured eye. Mice were anesthetized and a SDF-1-neutralizing antibody (MAB310, R&D Systems) or CXCR4-neutralizing antibody (2B11, BD Pharmagin) was injected intravitreally (2 μl total volume) to achieve a final concentration of 1 μg/μl for the SDF-1 antibody and 10 μg/μl for the CXCR4 antibody. For both antibodies, a 36-gauge needle and Hamilton syringe were used for the administration of the antibodies. Cohorts were given weekly booster injections for four weeks. For the intimal hyperplasia model a timed-release disc (Innovative Research of America, Sarasota Fla.) was surgically implanted adjacent to the vein graft at the time of surgery. The discs are engineered to release 5 μg antibody per day for up to one month. In the tumor neovascularization model antibodies were resuspended at 0.5 μg/μl in sterile PBS. 25 μg in 50 μl were then injected with a 0.5 cc tuberculin syringe every other day at the test site for the entire course of the experiment.

Primate: Monkey eyes were subjected to one of the following antibody treatment regimes for a period of one month: 1) no treatment to serve as the neovascularization positive control; 2) weekly (starting on the day of laser treatment) intravitreal injection of isotype-control antibody (50 μl of 1 μg/μl nonspecific IgG per injection) only to normal non-lasered eyes to serve as controls for changes to the retina induced by IgG alone; 3) normal untreated, unmanipulated eyes; 4) AAV2-VEGF plus laser photocoagulation and weekly anti-SDF-1 antibody (50 μl of 1 μg/μl of MAB310, R&D Systems, per injection) and the actual test cohort. Blood was drawn at weekly intervals and after eight weeks, animals were euthanized and extensive toxicology and necropsy were performed. Treated and untreated eyes were fixed in formalin, sectioned and stained with Hematoxilin and Eosin (H+E) for retinal structure and with antibody to the endothelial marker CD31 to definitively identify blood vessels.

In order to mobilize CD133⁺/CXCR4⁺/DsRed⁺ cells from the bone marrow, C57B1/6 mice were injected with 100 ng of sKitL (Peprotech). To inhibit SDF-1α or CXCR4 in vivo to block the mobilization of CD133⁺/CXCR4⁺/DsRed⁺ cells, cohorts of mice were injected intravenously with either 20 μg antibody to CXCR4 (clone 2B11) or 20 μg antibody to SDF-1 (clone 79014.111) in conjunction with 100 ng of sKitL. Control cohorts were injected with IgG isotype antibody or PBS. Peripheral blood was analyzed for the percentage CD133⁺CXCR4⁺ cells.

Images were obtained using a laser scanning spectral confocal microscope (TCS SP2; Leica Microsystems Heidelberg GmbH, Wetzlar, Germany). Quantification of the contribution of BM-derived DsRed+ cells was carried out modeling a previously described method (Banin et al., 2006. Invest Ophthalmol V is Sci 47:2125-2134.). In brief, confocal image montages of the entire retina (10× magnification) were used to quantify the area of vascular contribution by BM-derived DsRed⁺ cells, SMA⁺ cells, and colocalized BM-derived DsRed⁺ cells/SMA⁺ cells. The total area was calculated by carefully delineating the avascular zones in the retina of FITC Dextran perfused retinas and calculating the total area using Volocity Image Analysis Software. Similarly, the area of BM-derived DsRed⁺ cells, SMA⁺ cells, and BM-derived DsRed⁺ cells/SMA⁺ cells was calculated by using confocal image montages of the entire retina. Selected regions were then summed to generate total area of BM-derived DsRed⁺ cells. Student's t test was used to statistically compare the different experimental groups. Three observers blinded to experimental group calculated the number of DsRed⁺ cells in these images, and the resulting values from each image were averaged. The reported values represent the mean of these mean counts. For analysis, n=6 for each condition.

### Example 21

### Controlling Bone Marrow Cell Migration to Tumors and Tumor-Associated Vasculature

To study mechanisms responsible for governing BM contribution in postnatal neovasculogenesis, models were chosen based on observed differences in their levels of BM contribution during neovasculogenesis (FIG. 5). As one model system, the previously developed murine model of proliferative retinopathy demonstrating widespread BM-derived vessel contribution to the neovascularization process (Grant, M. B., et al. Nat Med 8: 607-612, 2002; Butler, J. M., et al. J Clin Invest 115, 86-93, 2005) was used. In addition, tumor neovascularization models were tested that have shown differing levels of BM cell migration to the tumor mass as well as integration into tumor-associated vasculature including Lewis lung carcinoma (LLC) and melanoma (B16) (De Palma, M., et al., Nat Med 9, 789-795, 2003; Purhonen, S., et al. Proc Natl Acad Sci USA 105: 6620-6625, 2008). A novel technique was used in which combinations of these models were established in the same mice. Individual mice demonstrating durable GFP⁺ or DsRed⁺ BM engraftment were subjected to either retinal injury and LLC inoculation or LLC and B16 inoculation (in contralateral limbs). GFP⁺ and DsRed⁺ chimeric mice showed no differences in BM contribution. Use of this technique allowed the tracking of the fate of BM-derived cells in different neovascularization models in single mice with similar engraftment chimerism, age, treatment and housing environment, thus controlling for potential experimental variables that may confound overall data output and interpretation.

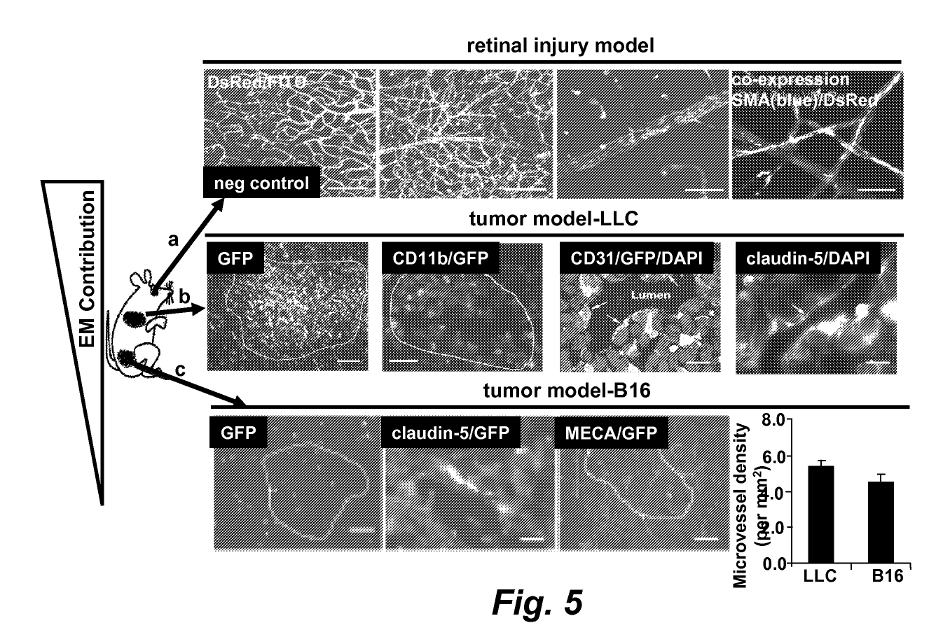

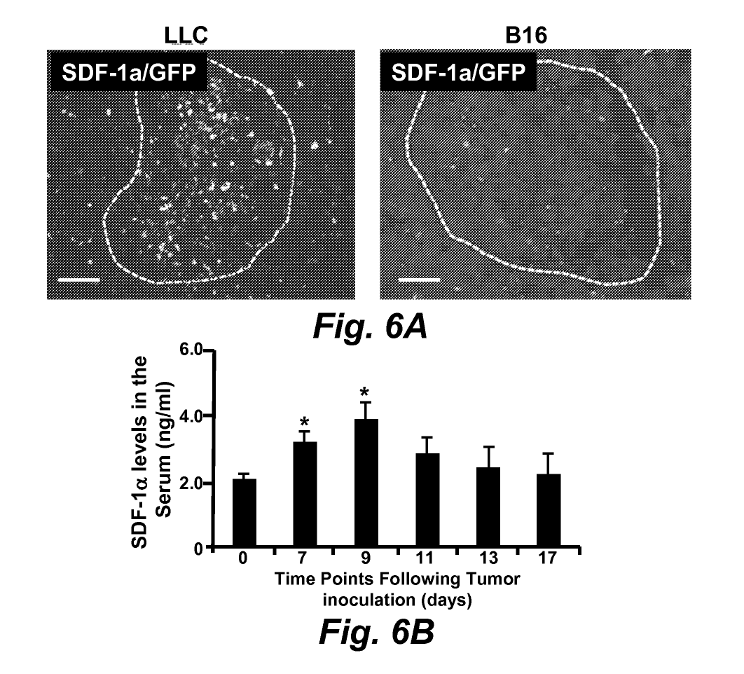

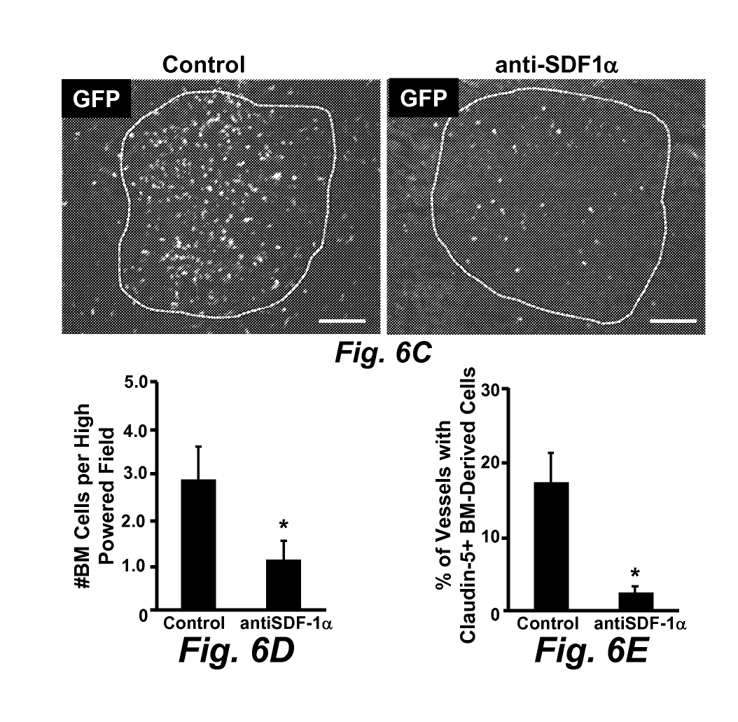

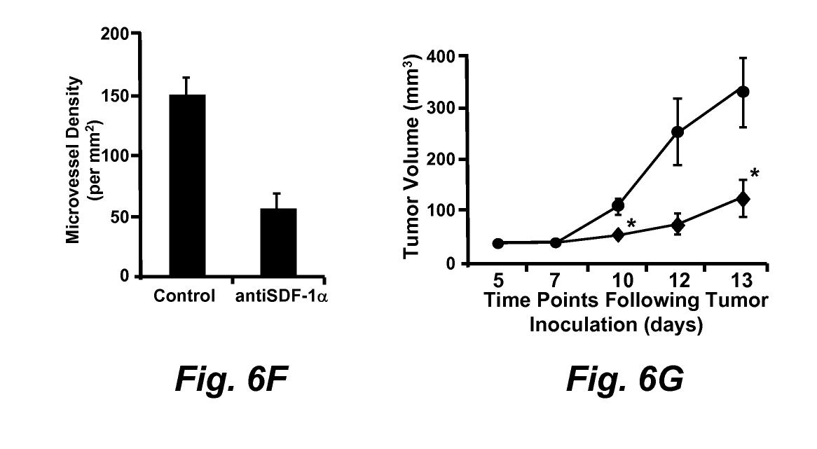

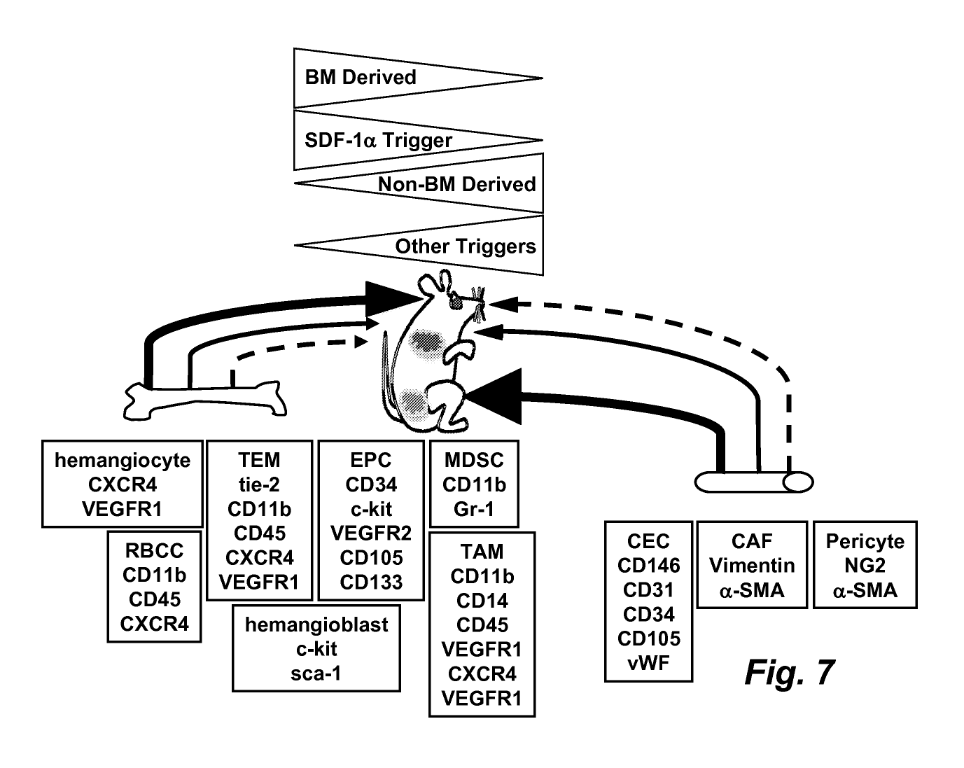

Analysis of these mice confirmed the spectrum of BM contribution across the different models, regardless of which combination was used. The retinal injury model provided the highest levels, generating vessels substantially comprised of functional BM-derived cells that co-expressed α-smooth muscle actin (SMA; FIG. 5a) (Grant, M. B., et al. Nat Med 8, 607-612, 2002; Butler, J. M., et al. J Clin Invest 115, 86-93, 2005). In all LLC tumors, recruitment of BM cells was observed, with the majority of cells found throughout the tumor mass (FIG. 5b). Immunofluorescent staining showed that these cells were mainly CD11b⁺ myelomonocytic cells, which are known to promote neovascularization through a paracrine mechanism as previously reported. Analysis of tumor sections for BM-derived cells co-expressing the endothelial marker, platelet endothelial cell adhesion molecule 1 (PECAM-1, CD31), found BM-derived cells with endothelial phenotype lining lumens of tumor-associated vasculature. Physical integration into endothelial linings was detected by staining for claudin-5, a tight-junction protein associated with endothelial cells. Primarily BM-derived vessels were not observed as in the retinal injury model, however the percent of tumor-associated vasculature containing at least one BM-derived cell, expressing claudin-5, per vessel section was approximately 17±4% (control in FIG. 6e). These findings indicate that BM contribution in LLC tumor-associated vasculature occurs through physical integration in a process that more closely resembles angiogenesis rather than whole blood vessel vasculogenesis. B16 tumors recruited significantly less BM cells to the tumor mass with no contribution to tumor-associated vasculature as shown using claudin-5 staining (FIG. 5c). Interestingly, even without apparent BM involvement, B16 tumors were capable of robust growth. Therefore, these tumors were still capable of neovascularization. When B16 tumors were stained for MECA32, many tumor-associated blood vessels were observed, albeit with no BM contribution. When similar staining was performed on LLC tumors from contralateral limbs and blood vessel density quantified, it was observed that both tumor types had statistically similar blood vessel densities (FIG. 5c). The gradation of BM contribution observed in these models suggests that neovascularization occurs through redundant mechanisms that may or may not involve BM. Alternate redundant mechanisms utilizing local angiogenesis or non-BM-derived endothelial elements, such as circulating endothelial cells (CECs), carcinoma associated fibroblasts (CAFs) or pericytes, migrating to the tumor site and participating in neovasculogenesis are likely candidates (Wels, J., Kaplan, et al., Genes Dev 22, 559-574, 2008; Dome, B., et al. Circulating endothelial cells, bone marrow-derived endothelial progenitor cells and proangiogenic hematopoietic cells in cancer: From biology to therapy. Crit. Rev Oncol Hematol (2008).

To identify factors that govern which of the redundant mechanisms (BM-derived or non BM-derived) are activated during post-natal neovascularization, we first concentrated on leukocyte trafficking factors known to modulate BM mobilization. Specifically, the local endogenous production of SDF-1α was focused on given its strong chemotactic effects on BM cells and previous studies suggesting its essential role in the ischemic eye model (Butler, J. M., et al. SDF-1 is both necessary and sufficient to promote proliferative retinopathy. J Clin Invest 115, 86-93, 2005; Petit, I. et al., Trends Immunol 28, 299-307, 2007). SDF-1α is constitutively expressed by the retinal-pigmented epithelium, thus serving as an internal positive control for SDF-1α staining (photoreceptor layer (PRL)). Following laser-induced retinal ischemic injury (in the right eye), an immediate upregulation of SDF-1α was observed in the ganglion cell layer (GCL) in as little as 1-hour following ischemic injury. Unmanipulated control (left eyes) and 0-hour eyes (pre-laser treatment) showed non-detectable levels of SDF-1α expression. Kinetic ELISA analysis of SDF-1α in the vitreal space showed significant increases in SDF-1α levels from 1 to 12-hours post-laser injury prior to returning to background levels. Interestingly, analysis of blood serum demonstrated increased levels of SDF-1α at 12-hours that continued until day 3. LLC tumors that showed BM contribution also demonstrated SDF-1α expression (FIG. 6a). In contrast, B16 melanomas showing little BM contribution demonstrated non-detectable levels of SDF-1α expression suggesting that the presence of SDF-1α in LLC tumors provides a trigger for BM incorporation into the tumor mass and tumor-associated blood vessels. Analysis of serum SDF-1α levels in mice inoculated with LLC tumors showed a marked increase by day 7 following inoculation returning to background levels by day 11 (FIG. 6b). Subsequent analysis of supernatants derived from in vitro cultured LLC cells showed non-detectable levels of SDF-1α protein, suggesting that endogenous SDF-1α is locally generated by cells recruited to the tumor environment. Together, these results point to a time dependent cascade between site-specific SDF-1α expression and serum levels, suggestive of an endogenous SDF-1α accumulation that promotes mobilization of BM cells to the peripheral blood and subsequent migration to the site of neovascularization.

Next, antibody-blocking studies were performed to determine the necessity of SDF-1α in BM-derived adult neovascularization. It was previously demonstrated that treatment with anti-SDF-1α neutralizing antibodies in the vitreous 1-day following ischemic retinal injury blocked recruitment and BM-derived neovascularization (Butler, J. M., et al. J Clin Invest 115, 86-93, 2005). When anti-SDF-1α antibodies were injected in LLC tumors, significantly lower BM recruitment was seen in the tumor mass (FIGS. 6c, 6d) as well as integrated within blood vessels (FIG. 6e). Microvessel density (MVD) was also significantly lower in anti-SDF-1α treated LLC tumors (FIG. 6f). The overall effect of anti-SDF-1α treatment on tumor size, which is a direct correlation to the extent of tumor neovascularization, was also assessed over a 2-week period and demonstrated that anti-SDF-1α generates significantly smaller tumors in comparison to controls (FIG. 6g). Together, these data strongly implicate SDF-1α as a major effector in the mechanism driving BM-derived neovasculogenesis across the spectrum of redundant neovascularization processes.

To better elucidate the mechanism of BM contribution to post-natal neovascularization, a cell population that directly participates in this process was identified. With the understanding that redundant mechanisms exist for postnatal neovascularization and that different model systems have differing levels of BM contribution, it was hypothesized that the retinal injury model would be the most robust for accomplishing this goal. Given the impact of SDF-1α on BM contribution, BM-derived cells that expressed CXCR4, the cognate receptor for SDF-1α, were focused on. To further enrich for vascular precursors, cells expressing CD133 were sorted for. Interestingly, further analysis of the BM-derived CD133⁺CXCR4⁺ population revealed the expression of a plurality of markers known to encompass the different phenotypically defined BM cells shown to participate in neovascularization (Dome, B., et al. Circulating endothelial cells, bone marrow-derived endothelial progenitor cells and proangiogenic hematopoietic cells in cancer: From biology to therapy. Crit. Rev Oncol Hematol 2008; Murdoch, C., et al., Nat Rev Cancer 8, 618-631, 2008).

To establish that BM-derived CD133⁺CXCR4⁺ cells directly participated in postnatal neovascularization, their levels in the peripheral blood following retinal ischemic injury were first assayed. A sustained increase of CD133⁺CXCR4⁺ cells was observed from 12-hours to 3-days in the blood of injured mice. Importantly, there was a distinct correlation between CD133⁺CXCR4⁺ numbers and levels of SDF-1α in blood serum where the highest levels of SDF-1α production preceded cell mobilization. In experiments where CD133⁺CXCR4⁺ cells were adoptively transferred to wild type recipient mice following ischemic injury, extensive contribution of CD133⁺CXCR4⁺ donor cells to the vasculature was observed. Moreover, this contribution could be prevented by anti-SDF-1α or anti-CXCR4⁺ treatment. All left eye negative controls showed no contribution from the CD133⁺CXCR4⁺ donor cells. Use of a model system with the highest BM contribution, allowed the identification of the CD133⁺CXCR4⁺ subpopulation as being enriched for cells capable of robust neovascularization in response to SDF-1α.

### Methods

Wild-type C57BL/6 mice were purchased from Charles River Laboratories. C57BL/6 mice that ubiquitously express DsRed.MST under the control of the chicken β-actin promoter and CMV enhancer were obtained from The Jackson Laboratory (Bar Harbor, Me.). The GFP⁺ mice are from STOCK Tg(GFPU)5Nagy/J mice (The Jackson Laboratory). All experimental procedures performed on animals were in accordance with the University of Florida institutional review board and Animal Care and Use Committee.

C57BL/6 chimeric mice were generated by irradiating recipient mice with 950 rads followed by retro-orbital sinus injection of 1×10⁶ whole BM cells enriched from GFP⁺ or DsRed⁺ mice as required. Mice were checked for multilineage engraftment using flow cytometry (FACSCalibur, BD Biosciences, San Jose, Calif.) 3 months post irradiation using monoclonal antibodies against CD11b, B220, CD4 and CD3e conjugated to FITC or PE (BD Pharmingen, San Diego, Calif.).

Mouse circulating mononuclear cells were labeled with the following monoclonal antibodies: PE-conjugated and FITC-conjugated CD133-specific (clone 13A4) and biotin-conjugated Tie-2 (TEK4) from eBiosciences (San Diego, Calif.); purified and FITC-conjugated CD184-specific (2B11/CXCR4), PE-conjugated CD45.2-specific (A20), PE-conjugated CD117-specific (2B8/c-kit), PE-conjugated Sca-1-specific (D7), PE-conjugated CD135 (A2F10.1), PE-conjugated CD11b-specific (M1/70), PE-conjugated CD31-specific (PECAM-1), PE-conjugated flk-1-specific (VEGF-R2), FITC-conjugated CD44 (IM7), FITC-conjugated CD106 (429NVLA-4), purified CD144 (1 ID4.1 VE-Cadherin) from BD Pharmingen; and CD150:ALEXA 647 from Serotec (Raleigh, N.C.). Secondary antibodies: streptavidin-PE and APC labeled goat anti-rat from BD Pharmingen.

The standard retinal neovascularization model was performed as described previously above. C57BL/6 chimeric mice were injected with 2×106 Lewis lung carcinoma cells (LLC, ATCC, Manassas, Va.) and/or melanoma cells (B16, ATCC) intramuscularly in hind limbs. Tumors were harvested for analysis once they reached a volume of between 500-600 mm3. In mice where retinal injury and LLC tumor models were combined, the injury was first established followed by LLC inoculation at day 28.

Peripheral blood from GFP⁺ or DsRed⁺ transgenic mice was isolated and the mononuclear cell fraction was collected with Ficoll Paque (Amersham Biosciences, Piscataway, N.J.) centrifugation purification. The mononuclear cells were washed in 5× volumes of PBS. The mononuclear layer was then resuspended in 100 μl of PBS and stained with monoclonal antibodies: rat anti-mouse monoclonal antibodies directed against CD133 (clone 13A4; FITC conjugate) and CD184/CXCR4 (clone 2B11), which was detected with an APC-conjugated goat anti-rat IgG antibody (BD Pharmingen). The cells were sorted using the FACSvantage SE for CD133⁺CXCR4⁺ (GFP⁺ or DsRed⁺) cells. One day following vessel photocoagulation, mice were anaesthetized and 1×10⁶ CD133⁺/CXCR4⁺ cells were infused into the retro-orbital sinus.

Immediately following laser photocoagulation, mice were anesthetized and SDF-1α-neutralizing antibody (MAB310, R&D Systems, Minneapolis, Minn.) or CXCR4-neutralizing antibody (2B11, BD Pharmingen) was injected intravitreally (2 μl total volume) to achieve a final concentration of 1 μg/μl for the anti-SDF-1α antibody and 10 μg/μl for the CXCR4 antibody. For both antibodies, a 36-gauge needle and Hamilton syringe were used for the administration of the antibodies. Cohorts were given weekly booster injections for four weeks.

To test the effects of SDF-1α on BM activity in cancer, slight modifications were made to the transplant model. After transplant and peripheral blood analysis at 3-months to confirm hematopoietic chimerism, mice were divided into 2 groups of 8. All mice received injections of 2×10⁶ LLC cells intramuscularly in hind limbs. One group of mice served as a control group while the other group received 25 μg of anti-SDF-1α antibodies (R&D Systems) in 20 μl PBS intratumorally each day. Tumors were measured daily using calipers. Volume measurements were calculated based on maximum width and length measurements.

Animals were sedated and perfused through the left ventricle with 4% paraformaldehyde (PFA) in PBS. Immediately following perfusion, eyes were enucleated by sliding curved forceps behind the eyeball and pulling the globe out, immersed in 4% PFA and placed in a 4° C. refrigerator overnight. Eyes were then processed and embedded in paraffin. Samples were sectioned at a thickness of 5 microns using a Microm microtome (Heidelberg, Germany) and picked up onto positive charged slides. Sections were left to air-dry overnight before being deparaffinized and appropriately retrieved for the antigens of interest. Neural retina required heat retrieval with 0.1M Citrate Buffer pH 6.0 for SDF-1α. Slides were washed in Tris Buffered Saline (TBS) and blocked with horse serum (Vector Laboratories) for 20 minutes. Goat anti-SDF-1α (SantaCruz Biotechnology, Santa Cruz Calif.) was applied at a dilution of 1:50 and incubated at 4° C. overnight. Slides were washed with TBS and stained for 1-hour in the dark with fluorescent secondary antibodies diluted at 1:200. Donkey anti-goat Alexafluor 594 was used for SDF-1α detection (Molecular Probes, Eugene Oreg.). Slides were again washed in TBS before coverslips were mounted with Vectashield containing 4′-6-diamidino-2-phenylindole (DAPI; Vector Laboratories).

One month following laser ablation, mice were deeply anesthetized intraperitoneally with avertin and perfused via the left ventricle with 3 ml of 4% PFA in PBS containing fluorescein isothio-cyanate-(FITC) or rhodamine isothio-cyanate (RITC) dextran (10 mg/ml, MW 70000, Sigma). Eyes were enucleated and placed in fresh 4% PFA for 60-minutes at room temperature. After washing in PBS, retinas were removed and flat mounted using Hardmount Vectashield without DAPI for imaging (Vector Laboratories).

Harvested tumor tissues were fixed overnight in 4% PFA and then equilibrated overnight in 18% sucrose. Fixed tissues were embedded in optimal cutting temperature (OCT) compound (Sakura Finetek USA, Torrance, Calif.), stored at −80° C. and later cryosectioned at 5 microns per section onto positively charged slides. Slides were air dried at room temperature overnight before staining. OCT was rinsed off of the sections using 1× Wash Solution (Dako, Carpenteria, Calif.) and then antigen retrieval was performed as needed. Slides were blocked in 3% horse serum for 20 minutes before the application of primary antibody overnight at 4° C. The following antibodies and titers were used: rat anti-CD11b (1:15; BD Pharmingen, San Diego Calif.), rat anti-MECA32 (1:10; BD Pharmingen), rabbit anti-claudin-5 (1:100; Novus Biologicals, Littleton Colo.), rat anti-CD31 (PECAM; 1:200; BD Pharmingen), and chicken anti-GFP (1:500, Abcam, Cambridge Mass.). Heat antigen retrieval with Citra buffer pH 6.0 for 25-minutes was required for optimal staining with claudin-5 and CD31. MECA32 stained slides were retrieved in Target Retrieval Solution (Dako) for 20-minutes at 95° C., followed by a 20-minute cool down at room temperature. The CD11b slides received 2-minutes of enzyme digestion (RTU Proteinase K, Dako) prior to staining. All slides were detected using 1:500 dilutions of species appropriate Alexa Fluor 594 antibodies raised in donkey (Molecular Probes) to allow simultaneous observation of GFP, either native or re-applied with antibody and detected with Alexa Fluor 488. In the case of CD11b, GFP could be directly visualized. However, GFP detection via antibody staining was needed when heat induced antigen retrieval methods were employed. Slides were mounted with Vectashield containing DAPI to allow for nuclear visualization. Positive control tissues and concentration matched Ig controls were included with each immunoassay.

The tissues that were collected for the detection of SDF-1α by ELISA included blood serum and vitreous fluid. Serum was collected by isolating peripheral blood from the retro-orbital plexus and allowing it to sit overnight at 4° C. Samples were centrifuged at 1,500 r.p.m. at 24-27° C. for 20-min and the serum was harvested in the form of a supernatant. Samples were immediately placed at −80° C. until time of analysis. Vitreous fluid was collected by anaesthetizing the mice and using a 36-gauge needle and Hamilton syringe. The needle was placed directly into the vitreous and 5 μl of vitreous fluid was removed. The fluid was placed in a 1.5 ml collection tube. 45 μl of PBS was added to the tube for a final volume of 50 μl. Samples were immediately placed at −80° C. until time of analysis. All samples were analyzed for SDF-1α using ELISA according to the manufacturer's instructions (R&D Systems). Tissues were analyzed using a laser scanning spectral confocal microscope (TCS SP2; Leica Microsystems, Bannockburn, Ill.) or an Olympus Provis immunofluorescence microscope (Olympus American, Melville, N.Y.). Statistical differences between different experimental groups were determined by one-way analysis of variance and student t-test. The reported values represent the mean±sem. A p-value less than 0.05 was considered significant.

### Example 22

### Anti-SDF-1 Ribozymes and SDF-1 Anti-Sense RNA Expression Constructs Decrease Migration of Cells that Revascularize the Eye

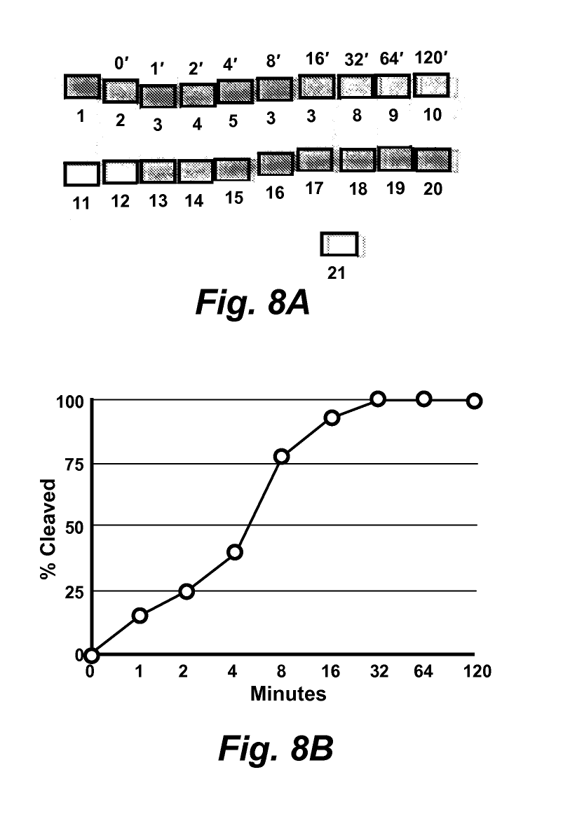

A self-cleaving hairpin ribozyme expression construct was created to target SDF-1. The efficacy of the anti-SDF-1 Ribozyme was tested in an in vitro cleavage assay to test destruction of SDF-1 mRNA. FIG. 8 shows the cleavage assay and the quantified results showing greater then 75% cleavage within 8 minutes.

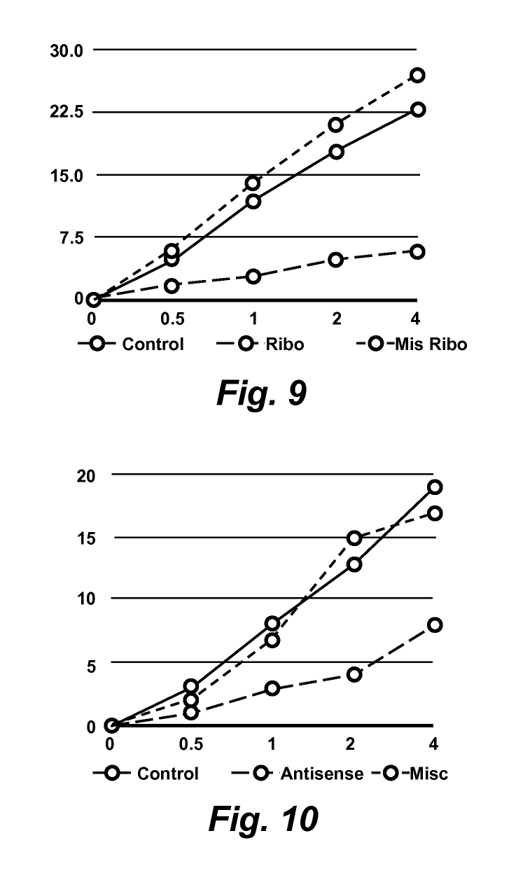

The anti-SDF-1 ribozyme construct was then used to infect a BM stromal cell line expressing SDF-1. Using a Boyden Chamber chemotaxis assay, the migration of GFP+Sca+Kit+ hematopoietic progenitors towards the SDF-1 expressing stroma was measured. As you can see the anti-SDF-1 Ribozyme expressing stroma significantly reduced progenitor homing—indicating the effectiveness of the ribozyme to reduce SDF-1 activity, FIG. 9. Referring to FIG. 9, for the control, BM Stroma expressing SDF-1 were placed in the bottom of a boyden chamber and 50 k Gfp+ HPC were placed in the top chamber. The chamber was incubated from 0-4 hours and the % of HPC that migrated to the bottom chamber was quantified. Ribo: Stroma was infected with the anti-SDF-1 ribozyme expression construct 48 hours before the migration assay. 50K HPC were added and migration quantified. M is Ribo: A scrambled sequence ribozyme non-specific ribozyme expression construct was used to infect the stromal cells 48 hrs before assay to serve as a control for alterations in migration due to the infection alone.

The same migration assay was used to test the efficacy of a SDF-1 anti-sense RNA expression construct from OpenBiosystems. The construct was once again cloned into the viral infection system and was used to infect the SDF-1 expressing stromal cell line. FIG. 10 shows that the SDF-1 anti-sense construct also significantly reduced migration of bone marrow multipotent progenitor cells (Sca-1+, cKit+ cells). Referring to FIG. 10, Control: BM Stroma expressing SDF-1 were placed in the bottom of a boyden chamber and 50 k Gfp+ HPC were placed in the top chamber. The chamber was incubated from 0-4 hours and the % of HPC that migrated to the bottom chamber was quantified. Antisense: Stroma was infected with the SDF-1 anti-sense RNA expression construct 48 hours before the migration assay. 50K HPC were added and migration quantified. Misc: A Nestin anti-sense RNA expression construct was used to infect the stromal cells 48 hrs before assay to serve as a specificity control for alterations in migration due to the anti-sense RNA expression alone. Nestin is not expressed in stromal cells so the anti-sense RNA should have minimal effect.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of reducing blood vessel formation in a neoplasia, the method comprising administering to a subject having a neoplasia a composition comprising an agent that binds SDF-1 and reduces SDF-1 biological activity in an amount effective to inhibit blood vessel formation in the neoplasia.

2. The method of claim 1, wherein the neoplasia is selected from the group consisting of: lung cancer, pancreatic cancer, melanoma, lymphoma, and leukemia.

3. The method of claim 1, wherein the agent that binds SDF-1 and reduces SDF-1 biological activity is an antibody that specifically binds SDF-1.

4. The method of claim 1, wherein administration of the composition to the subject decreases or halts growth of the neoplasia.

5. A method of reducing marrow cell mobilization in a subject having received chemotherapy, the method comprising administering to the subject being treated for a cancerous tumor at a particular site, a composition comprising an agent that binds SDF-1 and reduces SDF-1 biological activity in an amount effective to decrease or halt mobilization of marrow cells and cells that differentiate from marrow cells to the site after the subject has received chemotherapy.

6. The method of claim 5, wherein cells that differentiate from marrow cells comprise CD133⁺CXCR4⁺ cells and cells having surface expression of CD31 and vWF.

7. The method of claim 5, further comprising administering a vascular disrupting agent or an agent that reduces VEGF or Tie-2 biological activity to the subject.

8. The method of claim 5, wherein the composition is locally administered to a tumor.

9. The method of claim 5, wherein the administration is intratumoral.

10. The method of claim 5, wherein the composition is administered between about 7 and 60 days following chemotherapy.

11. The method of claim 5, wherein the composition is administered between about 1 and 28 days following chemotherapy.

12. The method of claim 5, wherein the composition is administered intravascularly.

13. A method of inhibiting cancerous tumor growth in a subject having a cancerous tumor, the method comprising administering to the subject an agent that binds to SDF-1 and reduces SDF-1 biological activity in an amount effective to decrease or block growth of the cancerous tumor in the subject.

14. The method of claim 13, wherein the agent that binds SDF-1 and reduces SDF-1 biological activity is an antibody that specifically binds SDF-1.

15. The method of claim 13, wherein the cancerous tumor growth is selected from the group consisting of: lung cancer, pancreatic cancer, melanoma, lymphoma, and leukemia.

16. The method of claim 13, further comprising administering an agent that inhibits VEGF or Tie-2 biological activity.

17. The method of claim 16, wherein the agent is bevacizumab.

18. The method of claim 14, wherein about 0.05-200 mg/kg of the SDF-1 specific antibody is administered to the subject.

19. A kit for the treatment of neoplasia or the prevention of a neoplasia relapse, the kit comprising a composition comprising a pharmaceutically acceptable carrier and SDF-1 antibody that specifically binds SDF-1 and blocks SDF-1 biological activity in an amount effective to inhibit neoplasia angiogenesis, and instructions for use.

20. The kit of claim 19, further comprising an agent that inhibits VEGF or Tie-2 biological activity.
